(19) United States
(12) Patent Application Publication
(10) Pub. No.: US 2024/0173285 A1
(43) Pub. Date: May 30, 2024

(54) COMPOSITIONS COMPRISING EPA AND METHODS OF USING THE SAME FOR TREATING AND/OR PREVENTING ENDOTHELIAL DYSFUNCTION IN A SUBJECT

(71) Applicants: Richard Preston MASON, Bridgewater, NJ (US); Amarin Pharmaceuticals Ireland Limited, Dublin (IE)

(72) Inventors: Richard Preston Mason, Bridgewater, NJ (US); Richard Louis Dunbar, Bridgewater, NJ (US)

(21) Appl. No.: 18/557,119

(22) PCT Filed: Apr. 29, 2022
(86) PCT No.: PCT/US2022/027119

§ 371 Date: Oct. 25, 2023

Related U.S. Application Data
(60) Provisional application No. 63/181,496, filed on Apr. 29, 2021.

(51) Int. Cl. A61K 31/202; A61K 31/20; A61K 31/201

(52) U.S. Cl. CPC A61K 31/202; A61K 31/20; A61K 31/201

(57) ABSTRACT

The present disclosure provides compositions and methods for treating and/or preventing endothelial dysfunction, increasing an activity of or an amount of heme oxygenase-1 (HO-1), and/or activating transcription factor Nrf2 (nuclear factor erythroid 2-related factor 2) in a subject in need thereof, comprising administering a daily dose of about 1 g to about 20 g of eicosapentaenoic acid or a derivative thereof to the subject.

### BACKGROUND

Cardiovascular disease is one of the leading causes of death in the United States and most European countries. It is estimated that over 70 million people in the United States alone suffer from a cardiovascular disease or disorder including but not limited to high blood pressure, coronary heart disease, dyslipidemia, congestive heart failure and stroke.

### SUMMARY

In various embodiments, the present disclosure provides compositions and methods for treating and/or preventing endothelial dysfunction, increasing an activity of or an amount of heme oxygenase-1 (HO-1), activating transcription factor Nrf2 (nuclear factor erythroid 2-related factor 2), activating antioxidant response elements (AREs), and/or cardiovascular-related diseases and disorders.

In some aspects, the present disclosure provides methods of treating and/or preventing endothelial dysfunction in a subject by administering to the subject a pharmaceutical composition comprising eicosapentaenoic acid (EPA) and/or a derivative thereof to provide a daily dose of about 1 g to about 20 g of the EPA and/or derivative thereof to the subject.

In some aspects, the present disclosure provides methods of increasing an activity of or an amount of HO-1 in a subject by administering to the subject a pharmaceutical composition comprising EPA or a derivative thereof to provide a daily dose of about 1 g to about 20 g of the EPA and/or derivative thereof to the subject.

In some aspects, the present disclosure provides methods of activating transcription factor Nrf2 in a subject by administering to the subject a pharmaceutical composition comprising EPA or a derivative thereof to provide a daily dose of about 1 g to about 20 g of the EPA and/or derivative thereof to the subject.

In some aspects, the present disclosure provides methods of activating AREs in a subject by administering to the subject a pharmaceutical composition comprising EPA or a derivative thereof to provide a daily dose of about 1 g to about 20 g of the EPA and/or derivative thereof to the subject.

In related aspects, the present disclosure further provides a method of assessing a suitability, dosage, and/or duration of the method described herein, the method comprising, prior to the administration, determining a concentration of HO-1 and/or a nucleotide sequence encoding HO-1, in bodily fluid or non-neural tissue obtained from the subject, and comparing the concentration with a corresponding concentration of HO-1 and/or an HO-1 encoding nucleotide sequence in a corresponding bodily fluid or non-neural tissue obtained from at least one control subject, wherein a reduced concentration between the subject and the control subject is used to determine the suitability, dosage, and/or duration.

In some embodiments, the pharmaceutical composition comprises at least about 80%, at least about 90%, at least about 95%, or at least about 96%, by weight of all fatty acids present, the eicosapentaenoic acid and/or derivative thereof.

In some embodiments, the pharmaceutical composition comprises no more than about 20%, no more than about 10%, no more than about 5%, or no more than about 3%, by weight of all fatty acids present, docosahexaenoic acid or derivatives thereof. In some embodiments, the pharmaceutical composition comprises no docosahexaenoic acid or derivatives thereof.

In some embodiments, the subject is administered about 1 g to about 20 g of the eicosapentaenoic acid and/or derivative thereof per day. In some embodiments, the subject is administered about 2 g of the eicosapentaenoic acid and/or derivative thereof per day. In some embodiments, the subject is administered about 4 g of the eicosapentaenoic acid and/or derivative thereof per day. In some embodiments, the subject is administered about 10 g of the eicosapentaenoic acid and/or derivative thereof per day. In some embodiments, the subject is administered about 15 g of the eicosapentaenoic acid and/or derivative thereof per day. In some embodiments, the subject is administered about 20 g of the eicosapentaenoic acid and/or derivative thereof per day.

In some embodiments, the subject is administered the pharmaceutical composition for a period of time between about 3 days to about 1 year. In some embodiments, the subject is administered the pharmaceutical composition for about 3 days. In some embodiments, the subject is administered the pharmaceutical composition for about 3 weeks. In some embodiments, the subject is administered the pharmaceutical composition for about 1 year.

In some embodiments, the eicosapentaenoic acid or derivative thereof comprises eicosatetraenoic acid ethyl ester (E-EPA) or icosapent ethyl.

In some embodiments, the derivative of EPA is at least one selected from the group consisting of 6-keto-prostaglandin F2 alpha (6k-PGF2a), thromboxane B3 (TXB3), 11-dehydro-thromboxane B3 (11-dTXB3), prostaglandin F3 alpha (PGF3a), prostaglandin E3 (PGE3), prostaglandin A3 (PGA3), prostaglandin D3 (PGD3), 2,3-dinor 11 beta-prostaglandin F3 alpha (2,3-dinor11bPGF3a), prostaglandin J3 (PGJ3), 15-deoxy-delta-12,14-prostaglandin J3 (15d-PGJ3), leukotriene B5 (LTB5), 20-hydroxy-leukotriene B5 (20-OH-LTB5), leukotriene C5 (LTC5), leukotriene D5 (LTD5), leukotriene E5 (LTE5), lipoxin A5 (LXA5), 5-oxo-eicosapentaenoic acid (5-oxo-EPE), 12-oxo-eicosapentaenoic acid (12-oxo-EPE), 15-oxo-eicosapentaenoic acid (15-oxo-EPE), 5-hydroxyeicosapentaenoic acid (5-HEPE), 8-hydroxyeicosapentaenoic acid (8-HEPE), 9-hydroxyeicosapentaenoic acid (9-HEPE), 11-hydroxyeicosapentaenoic acid (11-HEPE), 12-hydroxyeicosapentaenoic acid (12-HEPE), 15-hydroxyeicosapentaenoic acid (15-HEPE), 18-hydroxyeicosapentaenoic acid (18-HEPE), 19-hydroxyeicosapentaenoic acid (19-HEPE), 20-hydroxyeicosapentaenoic acid (20-HEPE), 5,6-epoxyeicosatetraenoic acid (5,6-EpETE), 8,9-epoxyeicosatetraenoic acid (8,9-EpETE), 11,12-epoxyeicosatetraenoic acid (11,12-EpETE), 14,15-epoxyeicosatetraenoic acid (14,15-EpETE), 5,6-dihydroxyeicosatetraenoic acid (5,6-diHETE), 8,9-dihydroxyeicosatetraenoic acid (8,9-diHETE), 11,12-dihydroxyeicosatetraenoic acid (11,12-diHETE), 14,15-dihydroxyeicosatetraenoic acid (14,15-diHETE), 17,18-dihydroxyeicosatetraenoic acid (17,18-diHETE), 17,18-epoxyeicosatetraenoic acid (17,18-EpETE), Resolvin E1 (RvE1), Resolvin E2 (RvE2), Resolvin E3 (RvE3), and Resolvin E4 (RvE4).

In some embodiments, the pharmaceutical composition further comprises a polyunsaturated fatty acid or a derivative thereof which is chemically distinct from the EPA or the derivative thereof.

In some embodiments, the polyunsaturated fatty acid is a long-chain fatty acid (LCFA).

In some embodiments, the LCFA is a long-chain polyunsaturated fatty acid (LCPUFA).

In related embodiments, the polyunsaturated fatty acid or derivative thereof is at least one selected from the group consisting of linoleic acid (FA 18:2, or LA), arachidonic acid (FA 20:4, or AA), docosapentaenoic acid (FA 22:5, or DPA), docosahexaenoic acid (FA 22:6, or DHA), a linoleic acid derivative (LA derivative), an arachidonic acid derivative (AA derivative), and a docosahexaenoic acid derivative (DHA derivative).

In some embodiments, the LA derivative is 9-hydroxyoctadecadienoic acid (9-HODE), 13-hydroxyoctadecadienoic acid (13-HODE), or both.

In some embodiments, the AA derivative is at least one selected from the group consisting of 6-keto-prostaglandin F1 alpha (6k-PGF1a), thromboxane B2 (TXB2), 11-dehydro-thromboxane B2 (11-dTXB2), prostaglandin F2 alpha (PGF2a), prostaglandin E2 (PGE2), prostaglandin A2 (PGA2), prostaglandin D2 (PGD2), 2,3-dinor 11 beta-prostaglandin F2 alpha (2,3-dinor11bPGF2a), prostaglandin J2 (PGJ2), 15-deoxy-delta-12,14-prostaglandin J2 (15d-PGJ2), leukotriene B4 (LTB4), 20-hydroxy-leukotriene B4 (20-OH-LTB4), leukotriene C4 (LTC4), leukotriene D4 (LTD4), leukotriene E4 (LTE4), lipoxin A4 (LXA4), 5-oxo-eicosatetraenoic acid (5-oxo-ETE), 12-oxo-eicosatetraenoic acid (12-oxo-ETE), 15-oxo-eicosatetraenoic acid (15-oxo-ETE), 5-hydroxyeicosatetraenoic acid (5-HETE), 8-hydroxyeicosatetraenoic acid (8-HETE), 9-hydroxyeicosatetraenoic acid (9-HETE), 11-hydroxyeicosatetraenoic acid (11-HETE), 12-hydroxyeicosatetraenoic acid (12-HETE), 15-hydroxyeicosatetraenoic acid (15-HETE), 18-hydroxyeicosatetraenoic acid (18-HETE), 19-hydroxyeicosatetraenoic acid (19-HETE), 20-hydroxyeicosatetraenoic acid (20-HETE), 5,6-epoxyeicosatrienoic acid (5,6-EET), 8,9-epoxyeicosatrienoic acid (8,9-EET), 11,12-epoxyeicosatrienoic acid (11,12-EET), 14,15-epoxyeicosatrienoic acid (14,15-EET), 5,6-dihydroxyeicosatrienoic acid (5,6-diHET), 8,9-dihydroxyeicosatrienoic acid (8,9-diHET), 11,12-dihydroxyeicosatrienoic acid (11,12-diHET), 14,15-dihydroxyeicosatrienoic acid (14,15-diHET), and 12-hydroxyheptadecatrenoic acid (12-HHTrE).

In some embodiments, the DHA derivative is at least one selected from the group consisting of 14-hydroxydocosahexaenoic acid (14-HDoHE), 17-hydroxydocosahexaenoic acid (17-HDoHE), and Resolvin D1 (RvD1).

In some embodiments, administration of the pharmaceutical composition reduces platelet aggregation, reduces inflammation, increases nitric oxide (NO) bioavailability, reduces a risk for thrombosis, and/or reduces a risk for atherosclerosis in the subject.

In some embodiments, administration of the pharmaceutical composition reduces a risk for or treats sepsis in the subject.

In some embodiments, administration of the pharmaceutical composition reduces a risk for or treats acute respiratory distress syndrome (ARDS) in the subject.

### BRIEF DESCRIPTION OF THE DRAWINGS

FIG. 1 is a diagram showing a summary of heme oxygenase-1 enzymatic pathway. The catabolism of heme by HMOX-1 produces biliverdin, CO, and free iron (Fe²⁺), which together facilitate the cytoprotective effects of HMOX-1.

FIG. 2 shows Volvano plots of IL-6 vs. vehicle (panel A), EPA+IL-6 vs. IL-6 (panel B), and DHA+IL-6 vs. IL-6 (panel C). All points above the horizontal line are considered significant (p<0.05).

FIG. 3 shows normalized protein abundance (top panel) and relative fold change differences (bottom panel) of all proteins detected in proteomic analysis. In the top heat map, the colors indicate normalized abundance of each protein signature. Mass spectra intensity values for each protein signature were log₁₀-transformed and then normalized across each treatment to fit on the same scale (shown on the right). In the bottom heat map, the colors indicate relative fold change increase (green) or decrease (purple) relative to IL-6 or vehicle. Each row corresponds to a unique protein.

FIG. 4 is a schematic view of paracellular monocyte transmigration. ICAM-1, Intercellular Adhesion Molecule-1; PECAM-1. ICAM-1, intercellular adhesion molecule-1; VCAM, vascular cell adhesion molecular-1; LBRC, lateral border recycling compartment.

FIG. 5 is a schematic illustration of the ONOO— assay. The nanosensor is used to quantitate endothelial NO and ONOO— release in real time and is made by depositing a sensing material on the tip of carbon fiber with a diameter of only 0.5 μm. The fiber is sealed with nonconductive epoxy and electrically connected to wires (gold, copper) with conductive silver epoxy. Conductive films of polymeric Ni(II) tetrakis (3-methoxy-4-hydroxyphenyl) porphyrin and Mn(III) paracyclophanylporphyrin were used for the NO and ONOO— sensors, respectively.

FIGS. 6A-6C show dot plots of significantly modulated pathways by IL-6 vs. vehicle (FIG. 6A), EPA+IL-6 vs. IL-6 (FIG. 6B), and AA+IL-6 vs. IL-6 (FIG. 6C). The pathways are organized by the number of significantly modulated proteins in each pathway (gene ratio) and the p-adjusted value, with the top row indicating the pathway that “scores” the highest is listed first.

FIG. 7 is an illustration showing therapeutic mechanisms regarding heme oxygenases such as HO-1.

FIG. 8 is an illustration showing mechanisms of EPA and oxylipin metabolites' induction of heme oxygenases such as HO-1.

### DETAILED DESCRIPTION

While the present disclosure is capable of being embodied in various forms, the description below of several embodiments is made with the understanding that the present disclosure is to be considered as an exemplification of the invention and is not intended to limit the invention to the specific embodiments illustrated. Headings are provided for convenience only and are not to be construed to limit the invention in any manner. Embodiments illustrated under any heading may be combined with embodiments illustrated under any other heading.

The use of numerical values in the various quantitative values specified in this application, unless expressly indicated otherwise, are stated as approximations as though the minimum and maximum values within the stated ranges were both preceded by the word “about.” It is to be understood, although not always explicitly stated, that all numerical designations are preceded by the term “about.” It is to be understood that such range format is used for convenience and brevity and should be understood flexibly to not only include numerical values explicitly specified as limits of a range, but also to include all individual numerical values or sub-ranges encompassed within that range as if each numerical value and sub-range is explicitly specified. For example, a ratio in the range of about 1 to about 200 should be understood to not only include the explicitly recited limits of about 1 and about 200, but also to include individual ratios such as about 2, about 3, and about 4, and sub-ranges such as about 10 to about 50, about 20 to about 100, and so forth. It also is to be understood, although not always explicitly stated, that the ranges described herein are merely exemplary and that equivalents of such are known in the art.

Also, the disclosure of ranges is intended as a continuous range including every value between the minimum and maximum values recited as well as any ranges that can be formed by such values. Also disclosed herein are any and all ratios (and ranges of any such ratios) that can be formed by dividing a disclosed numeric value into any other disclosed numeric value. Accordingly, the skilled person will appreciate that many such ratios, ranges, and ranges of ratios can be unambiguously derived from the numerical values presented herein and in all instances such ratios, ranges, and ranges of ratios represent various embodiments of the present disclosure.

### Definitions

The term “about,” as used herein when referring to a measurable value such as an amount or concentration and the like, is meant to encompass variations of 20%, 10%, 5%, 1%, 0.5%, or even 0.1% of the specified amount.

The term “derivative,” as used herein when referring to a fatty acid, is meant to encompass any modified form of the fatty acid that was derived, for example, by a chemical reaction from the fatty acid in free acid form (i.e., terminal carboxylic acid functional group). Non-limiting examples of fatty acid derivatives as used herein include oxidative metabolites of fatty acids such as oxylipins; alkyl esters such as methyl esters, propyl esters, butyl esters, or ethyl esters; a salt of the fatty acid such as a lithium, sodium, or potassium salt; or a glyceride form of the fatty acid such as a mono-, di-, or triglyceride fatty acid.

The term “statistical significance,” as used herein refers to a result from data generated by testing or experimentation is not likely to occur randomly or by chance, but is instead likely to be attributable to a specific cause. Statistical significance is evaluated from a calculated probability (p-value), where the p-value is a function of the means and standard deviations of the data samples and indicates the probability under which a statistical result occurred by chance or by sampling error. A result is considered statistically significant if the p-value is 0.05 or less, corresponding to a confidence level of 95%.

The term “control subject,” as used herein refers to any subject used as a basis for comparison to the test subject. A control subject includes, but is not limited to, any subject who has not been administered the composition, administered a composition other than the test composition (e.g., Lovaza® comprised of 365 mg of E-EPA and 375 mg of E-DHA), or administered a placebo. A disease control (e.g., control person) could also be a person without the disease.

### Compositions

In some embodiments, a composition for use in methods of the disclosure comprises eicosapentaenoic acid in its free acid form, or a pharmaceutically acceptable ester, derivative, conjugate, or salt thereof, or mixtures of any of the foregoing, collectively referred to herein as “EPA.” The term “pharmaceutically acceptable” in the present context means that the substance in question does not produce unacceptable toxicity to the subject or interaction with other components of the composition. In one embodiment, derivatives of EPA include, but are not limited to, methyl, ethyl, or other alkyl esters, re-esterified monoglycerides, re-esterified diglycerides, and re-esterified triglycerides or mixtures thereof.

In some embodiments, the EPA comprises an eicosapentaenoic acid ester. In some embodiments, the EPA comprises a C₁-C₅ alkyl ester of eicosapentaenoic acid. In some embodiments, the EPA comprises eicosapentaenoic acid ethyl ester (E-EPA), eicosapentaenoic acid methyl ester, eicosapentaenoic acid propyl ester, or eicosapentaenoic acid butyl ester. In the present disclosure, eicosapentaenoic acid ethyl ester, icosapent ethyl, and E-EPA are referenced interchangeably.

In some embodiments, the EPA is in the form of ethyl-EPA, methyl-EPA, lithium EPA, mono-, di- or triglyceride EPA or any other ester or salt of EPA, or the free acid form of EPA. The EPA may also be in the form of a 2-substituted derivative or other derivative which slows down its rate of oxidation but does not otherwise change its biological action to any substantial degree. Where any particular form of EPA (e.g., eicosapentaenoic acid ethyl ester, icosapent ethyl, or E-EPA) is referred to throughout this application, any pharmaceutically acceptable derivative of EPA can be substituted in its place including icosapent methyl or eicosapentaenoic acid in free acid form. In one embodiment, such derivatives of EPA are administered daily in amounts containing the same number of moles of EPA contained in 1-20 grams of icosapent ethyl.

In some embodiments, a composition of the disclosure is administered to a subject in an amount sufficient to provide a daily dose of EPA of about 1 mg to about 40,000 mg, about 10 mg to about 30,000 mg, about 20 mg to about 20,000 mg, about 25 mg to about 10,000 mg, about 50 mg to about 5000 mg, about 75 mg to about 2500 mg, or about 100 mg to about 1000 mg, for example about 75 mg, about 100 mg, about 125 mg, about 150 mg, about 175 mg, about 200 mg, about 225 mg, about 250 mg, about 275 mg, about 300 mg, about 325 mg, about 350 mg, about 375 mg, about 400 mg, about 425 mg, about 450 mg, about 475 mg, about 500 mg, about 525 mg, about 550 mg, about 575 mg, about 600 mg, about 625 mg, about 650 mg, about 675 mg, about 700 mg, about 725 mg, about 750 mg, about 775 mg, about 800 mg, about 825 mg, about 850 mg, about 875 mg, about 900 mg, about 925 mg, about 950 mg, about 975 mg, about 1000 mg, about 1025 mg, about 1050 mg, about 1075 mg, about 1100 mg, about 1125 mg, about 1150 mg, about 1175 mg, about 1200 mg, about 1225 mg, about 1250 mg, about 1275 mg, about 1300 mg, about 1325 mg, about 1350 mg, about 1375 mg, about 1400 mg, about 1425 mg, about 1450 mg, about 1475 mg, about 1500 mg, about 1525 mg, about 1550 mg, about 1575 mg, about 1600 mg, about 1625 mg, about 1650 mg, about 1675 mg, about 1700 mg, about 1725 mg, about 1750 mg, about 1775 mg, about 1800 mg, about 1825 mg, about 1850 mg, about 1875 mg, about 1900 mg, about 1925 mg, about 1950 mg, about 1975 mg, about 2000 mg, about 2025 mg, about 2050 mg, about 2075 mg, about 2100 mg, about 2125 mg, about 2150 mg, about 2175 mg, about 2200 mg, about 2225 mg, about 2250 mg, about 2275 mg, about 2300 mg, about 2325 mg, about 2350 mg, about 2375 mg, about 2400 mg, about 2425 mg, about 2450 mg, about 2475 mg, about 2500 mg, about 2525 mg, about 2550 mg, about 2575 mg, about 2600 mg, about 2625 mg, about 2650 mg, about 2675 mg, about 2700 mg, about 2725 mg, about 2750 mg, about 2775 mg, about 2800 mg, about 2825 mg, about 2850 mg, about 2875 mg, about 2900 mg, about 2925 mg, about 2950 mg, about 2975 mg, about 3000 mg, about 3025 mg, about 3050 mg, about 3075 mg, about 3100 mg, about 3125 mg, about 3150 mg, about 3175 mg, about 3200 mg, about 3225 mg, about 3250 mg, about 3275 mg, about 3300 mg, about 3325 mg, about 3350 mg, about 3375 mg, about 3400 mg, about 3425 mg, about 3450 mg, about 3475 mg, about 3500 mg, about 3525 mg, about 3550 mg, about 3575 mg, about 3600 mg, about 3625 mg, about 3650 mg, about 3675 mg, about 3700 mg, about 3725 mg, about 3750 mg, about 3775 mg, about 3800 mg, about 3825 mg, about 3850 mg, about 3875 mg, about 3900 mg, about 3925 mg, about 3950 mg, about 3975 mg, about 4000 mg, about 4025 mg, about 4050 mg, about 4075 mg, about 4100 mg, about 4125 mg, about 4150 mg, about 4175 mg, about 4200 mg, about 4225 mg, about 4250 mg, about 4275 mg, about 4300 mg, about 4325 mg, about 4350 mg, about 4375 mg, about 4400 mg, about 4425 mg, about 4450 mg, about 4475 mg, about 4500 mg, about 4525 mg, about 4550 mg, about 4575 mg, about 4600 mg, about 4625 mg, about 4650 mg, about 4675 mg, about 4700 mg, about 4725 mg, about 4750 mg, about 4775 mg, about 4800 mg, about 4825 mg, about 4850 mg, about 4875 mg, about 4900 mg, about 4925 mg, about 4950 mg, about 4975 mg, about 5000 mg, about 5025 mg, about 5050 mg, about 5075 mg, about 5100 mg, about 5125 mg, about 5150 mg, about 5175 mg, about 5200 mg, about 5225 mg, about 5250 mg, about 5275 mg, about 5300 mg, about 5325 mg, about 5350 mg, about 5375 mg, about 5400 mg, about 5425 mg, about 5450 mg, about 5475 mg, about 5500 mg, about 5525 mg, about 5550 mg, about 5575 mg, about 5600 mg, about 5625 mg, about 5650 mg, about 5675 mg, about 5700 mg, about 5725 mg, about 5750 mg, about 5775 mg, about 5800 mg, about 5825 mg, about 5850 mg, about 5875 mg, about 5900 mg, about 5925 mg, about 5950 mg, about 5975 mg, about 6000 mg, about 6025 mg, about 6050 mg, about 6075 mg, about 6100 mg, about 6125 mg, about 6150 mg, about 6175 mg, about 6200 mg, about 6225 mg, about 6250 mg, about 6275 mg, about 6300 mg, about 6325 mg, about 6350 mg, about 6375 mg, about 6400 mg, about 6425 mg, about 6450 mg, about 6475 mg, about 6500 mg, about 6525 mg, about 6550 mg, about 6575 mg, about 6600 mg, about 6625 mg, about 6650 mg, about 6675 mg, about 6700 mg, about 6725 mg, about 6750 mg, about 6775 mg, about 6800 mg, about 6825 mg, about 6850 mg, about 6875 mg, about 6900 mg, about 6925 mg, about 6950 mg, about 6975 mg, about 7000 mg, about 7025 mg, about 7050 mg, about 7075 mg, about 7100 mg, about 7125 mg, about 7150 mg, about 7175 mg, about 7200 mg, about 7225 mg, about 7250 mg, about 7275 mg, about 7300 mg, about 7325 mg, about 7350 mg, about 7375 mg, about 7400 mg, about 7425 mg, about 7450 mg, about 7475 mg, about 7500 mg, about 7525 mg, about 7550 mg, about 7575 mg, about 7600 mg, about 7625 mg, about 7650 mg, about 7675 mg, about 7700 mg, about 7725 mg, about 7750 mg, about 7775 mg, about 7800 mg, about 7825 mg, about 7850 mg, about 7875 mg, about 7900 mg, about 7925 mg, about 7950 mg, about 7975 mg, about 8000 mg, about 8025 mg, about 8050 mg, about 8075 mg, about 8100 mg, about 8125 mg, about 8150 mg, about 8175 mg, about 8200 mg, about 8225 mg, about 8250 mg, about 8275 mg, about 8300 mg, about 8325 mg, about 8350 mg, about 8375 mg, about 8400 mg, about 8425 mg, about 8450 mg, about 8475 mg, about 8500 mg, about 8525 mg, about 8550 mg, about 8575 mg, about 8600 mg, about 8625 mg, about 8650 mg, about 8675 mg, about 8700 mg, about 8725 mg, about 8750 mg, about 8775 mg, about 8800 mg, about 8825 mg, about 8850 mg, about 8875 mg, about 8900 mg, about 8925 mg, about 8950 mg, about 8975 mg, about 9000 mg, about 9025 mg, about 9050 mg, about 9075 mg, about 9100 mg, about 9125 mg, about 9150 mg, about 9175 mg, about 9200 mg, about 9225 mg, about 9250 mg, about 9275 mg, about 9300 mg, about 9325 mg, about 9350 mg, about 9375 mg, about 9400 mg, about 9425 mg, about 9450 mg, about 9475 mg, about 9500 mg, about 9525 mg, about 9550 mg, about 9575 mg, about 9600 mg, about 9625 mg, about 9650 mg, about 9675 mg, about 9700 mg, about 9725 mg, about 9750 mg, about 9775 mg, about 9800 mg, about 9825 mg, about 9850 mg, about 9875 mg, about 9900 mg, about 9925 mg, about 9950 mg, about 9975 mg, about 10,000 mg, about 11,000 mg, about 12,000 mg, about 13,000 mg, about 14,000 mg, about 15,000 mg, about 16,000 mg, about 17,000 mg, about 18,000 mg, about 19,000 mg, or about 20,000 mg.

In some embodiments, EPA is present in a composition useful in accordance with methods of the disclosure in an amount of about 50 mg to about 5000 mg, about 75 mg to about 2500 mg, or about 100 mg to about 1000 mg, for example about 75 mg, about 100 mg, about 125 mg, about 150 mg, about 175 mg, about 200 mg, about 225 mg, about 250 mg, about 275 mg, about 300 mg, about 325 mg, about 350 mg, about 375 mg, about 400 mg, about 425 mg, about 450 mg, about 475 mg, about 500 mg, about 525 mg, about 550 mg, about 575 mg, about 600 mg, about 625 mg, about 650 mg, about 675 mg, about 700 mg, about 725 mg, about 750 mg, about 775 mg, about 800 mg, about 825 mg, about 850 mg, about 875 mg, about 900 mg, about 925 mg, about 950 mg, about 975 mg, about 1000 mg, about 1025 mg, about 1050 mg, about 1075 mg, about 1100 mg, about 1125 mg, about 1150 mg, about 1175 mg, about 1200 mg, about 1225 mg, about 1250 mg, about 1275 mg, about 1300 mg, about 1325 mg, about 1350 mg, about 1375 mg, about 1400 mg, about 1425 mg, about 1450 mg, about 1475 mg, about 1500 mg, about 1525 mg, about 1550 mg, about 1575 mg, about 1600 mg, about 1625 mg, about 1650 mg, about 1675 mg, about 1700 mg, about 1725 mg, about 1750 mg, about 1775 mg, about 1800 mg, about 1825 mg, about 1850 mg, about 1875 mg, about 1900 mg, about 1925 mg, about 1950 mg, about 1975 mg, about 2000 mg, about 2025 mg, about 2050 mg, about 2075 mg, about 2100 mg, about 2125 mg, about 2150 mg, about 2175 mg, about 2200 mg, about 2225 mg, about 2250 mg, about 2275 mg, about 2300 mg, about 2325 mg, about 2350 mg, about 2375 mg, about 2400 mg, about 2425 mg, about 2450 mg, about 2475 mg, about 2500 mg, about 2525 mg, about 2550 mg, about 2575 mg, about 2600 mg, about 2625 mg, about 2650 mg, about 2675 mg, about 2700 mg, about 2725 mg, about 2750 mg, about 2775 mg, about 2800 mg, about 2825 mg, about 2850 mg, about 2875 mg, about 2900 mg, about 2925 mg, about 2950 mg, about 2975 mg, about 3000 mg, about 3025 mg, about 3050 mg, about 3075 mg, about 3100 mg, about 3125 mg, about 3150 mg, about 3175 mg, about 3200 mg, about 3225 mg, about 3250 mg, about 3275 mg, about 3300 mg, about 3325 mg, about 3350 mg, about 3375 mg, about 3400 mg, about 3425 mg, about 3450 mg, about 3475 mg, about 3500 mg, about 3525 mg, about 3550 mg, about 3575 mg, about 3600 mg, about 3625 mg, about 3650 mg, about 3675 mg, about 3700 mg, about 3725 mg, about 3750 mg, about 3775 mg, about 3800 mg, about 3825 mg, about 3850 mg, about 3875 mg, about 3900 mg, about 3925 mg, about 3950 mg, about 3975 mg, about 4000 mg, about 4025 mg, about 4050 mg, about 4075 mg, about 4100 mg, about 4125 mg, about 4150 mg, about 4175 mg, about 4200 mg, about 4225 mg, about 4250 mg, about 4275 mg, about 4300 mg, about 4325 mg, about 4350 mg, about 4375 mg, about 4400 mg, about 4425 mg, about 4450 mg, about 4475 mg, about 4500 mg, about 4525 mg, about 4550 mg, about 4575 mg, about 4600 mg, about 4625 mg, about 4650 mg, about 4675 mg, about 4700 mg, about 4725 mg, about 4750 mg, about 4775 mg, about 4800 mg, about 4825 mg, about 4850 mg, about 4875 mg, about 4900 mg, about 4925 mg, about 4950 mg, about 4975 mg, or about 5000 mg.

In some embodiments, a composition useful in accordance with the disclosure contains no more than about 10%, no more than about 9%, no more than about 8%, no more than about 7%, no more than about 6%, no more than about 5%, no more than about 4%, no more than about 3%, no more than about 2%, no more than about 1%, or no more than about 0.5%, by weight, of docosahexaenoic acid (DHA) or derivatives thereof, if any. In some embodiments, a composition of the disclosure contains substantially no DHA or derivatives thereof. In some embodiments, a composition useful in the present disclosure contains no DHA and/or derivative thereof. In some embodiments, derivatives of DHA include, but are not limited to, methyl or other alkyl esters, re-esterified monoglycerides, re-esterified diglycerides, and re-esterified triglycerides or mixtures thereof.

In some embodiments, EPA comprises at least about 70%, at least about 80%, at least about 90%, at least about 95%, at least about 96%, at least about 97%, at least about 98%, at least about 99%, or at least about 100%, by weight, of all fatty acids present in a composition that is useful in methods of the present disclosure.

In some embodiments, the composition comprises at least 96% by weight of eicosapentaenoic acid ethyl ester and less than about 2% by weight of a preservative. In some embodiments, the preservative is a tocopherol such as all-racemic α-tocopherol.

In some embodiments, a composition useful in accordance with methods of the disclosure contains less than about 10%, less than about 9%, less than about 8%, less than about 7%, less than about 6%, less than about 5%, less than about 4%, less than about 3%, less than about 2%, less than about 1%, less than about 0.5% or less than about 0.25%, by weight of the total composition or by weight of the total fatty acid content, of any fatty acid other than EPA. Illustrative examples of a “fatty acid other than EPA” include linolenic acid (LA), arachidonic acid (AA), docosahexaenoic acid (DHA), alpha-linolenic acid (ALA), stearidonic acid (SDA), eicosatrienoic acid (ETA) and/or docosapentaenoic acid (DPA). In some embodiments, a composition useful in accordance with methods of the disclosure contains about 0.1% to about 4%, about 0.5% to about 3%, or about 1% to about 2%, by weight, of total fatty acids other than EPA and/or DHA. In one embodiment, fatty acids other than EPA include derivatives of those fatty acids. Derivatives of the fatty acids include, but are not limited to, methyl or other alkyl esters, re-esterified monoglycerides, re-esterified diglycerides, and re-esterified triglycerides or mixtures thereof of the fatty acids.

In some embodiments, a composition useful in accordance with the disclosure has one or more of the following features: (a) eicosapentaenoic acid ethyl ester represents at least about 96%, at least about 97%, or at least about 98%, by weight, of all fatty acids present in the composition; (b) the composition contains no more than about 4%, no more than about 3%, or no more than about 2%, by weight, of total fatty acids other than eicosapentaenoic acid ethyl ester; (c) the composition contains no more than about 0.6%, no more than about 0.5%, or no more than about 0.4% of any individual fatty acid other than eicosapentaenoic acid ethyl ester; (d) the composition has a refractive index (20° C.) of about 1.0 to about 2.0, about 1.2 to about 1.8, or about 1.4 to about 1.5; (e) the composition has a specific gravity (20° C.) of about 0.8 to about 1.0, about 0.85 to about 0.95, or about 0.9 to about 0.92; (f) the composition contains no more than about 20 ppm, no more than about 15 ppm, or no more than about 10 ppm heavy metals; (g) the composition contains no more than about 5 ppm, no more than about 4 ppm, no more than about 3 ppm, or no more than about 2 ppm arsenic; and/or (h) the composition has a peroxide value of no more than about 5 meq/kg, no more than about 4 meq/kg, no more than about 3 meq/kg, or no more than about 2 meq/kg.

In some embodiments, a composition for use in accordance with the disclosure comprises the EPA and at least one derivative of EPA. The derivative of EPA may be at least one selected from the group consisting of 6-keto-prostaglandin F2 alpha (6k-PGF2a), thromboxane B3 (TXB3), 11-dehydro-thromboxane B3 (11-dTXB3), prostaglandin F3 alpha (PGF3a), prostaglandin E3 (PGE3), prostaglandin A3 (PGA3), prostaglandin D3 (PGD3), 2,3-dinor 11 beta-prostaglandin F3 alpha (2,3-dinor11bPGF3a), prostaglandin J3 (PGJ3), 15-deoxy-delta-12,14-prostaglandin J3 (15d-PGJ3), leukotriene B5 (LTB5), 20-hydroxy-leukotriene B5 (20-OH-LTB5), leukotriene C5 (LTC5), leukotriene D5 (LTD5), leukotriene E5 (LTE5), lipoxin A5 (LXA5), 5-oxo-eicosapentaenoic acid (5-oxo-EPE), 12-oxo-eicosapentaenoic acid (12-oxo-EPE), 15-oxo-eicosapentaenoic acid (15-oxo-EPE), 5-hydroxyeicosapentaenoic acid (5-HEPE), 8-hydroxyeicosapentaenoic acid (8-HEPE), 9-hydroxyeicosapentaenoic acid (9-HEPE), 11-hydroxyeicosapentaenoic acid (11-HEPE), 12-hydroxyeicosapentaenoic acid (12-HEPE), 15-hydroxyeicosapentaenoic acid (15-HEPE), 18-hydroxyeicosapentaenoic acid (18-HEPE), 19-hydroxyeicosapentaenoic acid (19-HEPE), 20-hydroxyeicosapentaenoic acid (20-HEPE), 5,6-epoxyeicosatetraenoic acid (5,6-EpETE), 8,9-epoxyeicosatetraenoic acid (8,9-EpETE), 11,12-epoxyeicosatetraenoic acid (11,12-EpETE), 14,15-epoxyeicosatetraenoic acid (14,15-EpETE), 5,6-dihydroxyeicosatetraenoic acid (5,6-diHETE), 8,9-dihydroxyeicosatetraenoic acid (8,9-diHETE), 11,12-dihydroxyeicosatetraenoic acid (11,12-diHETE), 14,15-dihydroxyeicosatetraenoic acid (14,15-diHETE), 17,18-dihydroxyeicosatetraenoic acid (17,18-diHETE), 17,18-epoxyeicosatetraenoic acid (17,18-EpETE), Resolvin E1 (RvE1), Resolvin E2 (RvE2), Resolvin E3 (RvE3), and Resolvin E4 (RvE4).

In some embodiments, a composition for use in accordance with the disclosure comprises a polyunsaturated fatty acid or a derivative thereof that is chemically distinct from the EPA or the derivative thereof as previously described. In other embodiments, a composition for use in accordance with the disclosure further comprises a polyunsaturated fatty acid or a derivative thereof, in addition to the EPA or the derivative thereof as previously described.

As used herein, long-chain fatty acids are fatty acids having at least 18 carbon atoms.

In some embodiments, the polyunsaturated fatty acid is a long-chain fatty acid (LCFA). In some embodiments, the LCFA is a long-chain polyunsaturated fatty acid (LCPUFA).

In some embodiments, the polyunsaturated fatty acid or derivative thereof is at least one selected from the group consisting of linoleic acid (FA 18:2, or LA), arachidonic acid (FA 20:4, or AA), docosapentaenoic acid (FA 22:5, or DPA), docosahexaenoic acid (FA 22:6, or DHA), a linoleic acid derivative (LA derivative), an arachidonic acid derivative (AA derivative), and a docosahexaenoic acid derivative (DHA derivative).

Non-limiting examples of the LA derivative include 9-hydroxyoctadecadienoic acid (9-HODE), and 13-hydroxyoctadecadienoic acid (13-HODE).

Non-limiting examples of the AA derivative include 6-keto-prostaglandin F1 alpha (6k-PGF1a), thromboxane B2 (TXB2), 11-dehydro-thromboxane B2 (11-dTXB2), prostaglandin F2 alpha (PGF2a), prostaglandin E2 (PGE2), prostaglandin A2 (PGA2), prostaglandin D2 (PGD2), 2,3-dinor 11 beta-prostaglandin F2 alpha (2,3-dinor11bPGF2a), prostaglandin J2 (PGJ2), 15-deoxy-delta-12,14-prostaglandin J2 (15d-PGJ2), leukotriene B4 (LTB4), 20-hydroxy-leukotriene B4 (20-OH-LTB4), leukotriene C4 (LTC4), leukotriene D4 (LTD4), leukotriene E4 (LTE4), lipoxin A4 (LXA4), 5-oxo-eicosatetraenoic acid (5-oxo-ETE), 12-oxo-eicosatetraenoic acid (12-oxo-ETE), 15-oxo-eicosatetraenoic acid (15-oxo-ETE), 5-hydroxyeicosatetraenoic acid (5-HETE), 8-hydroxyeicosatetraenoic acid (8-HETE), 9-hydroxyeicosatetraenoic acid (9-HETE), 11-hydroxyeicosatetraenoic acid (11-HETE), 12-hydroxyeicosatetraenoic acid (12-HETE), 15-hydroxyeicosatetraenoic acid (15-HETE), 18-hydroxyeicosatetraenoic acid (18-HETE), 19-hydroxyeicosatetraenoic acid (19-HETE), 20-hydroxyeicosatetraenoic acid (20-HETE), 5,6-epoxyeicosatrienoic acid (5,6-EET), 8,9-epoxyeicosatrienoic acid (8,9-EET), 11,12-epoxyeicosatrienoic acid (11,12-EET), 14,15-epoxyeicosatrienoic acid (14,15-EET), 5,6-dihydroxyeicosatrienoic acid (5,6-diHET), 8,9-dihydroxyeicosatrienoic acid (8,9-diHET), 11,12-dihydroxyeicosatrienoic acid (11,12-diHET), 14,15-dihydroxyeicosatrienoic acid (14,15-diHET), and 12-hydroxyheptadecatrenoic acid (12-HHTrE).

Non-limiting examples of the DHA derivative include 14-hydroxydocosahexaenoic acid (14-HDoHE), 17-hydroxydocosahexaenoic acid (17-HDoHE), and Resolvin D1 (RvD1). In some embodiments, a composition for use in accordance with the disclosure comprises E-EPA and one or more oxylipins. Exemplary oxylipins include, but are not limited to, the above-listed EPA derivatives, LA derivatives, AA derivatives, and DHA derivatives.

In some embodiments, a composition for use in accordance with the disclosure is a self-emulsifying composition. In some embodiments, the self-emulsifying composition comprises at least one compound selected from the group consisting of an omega-3 fatty acid and derivative thereof (e.g., pharmaceutically acceptable salt and/or ester). In some embodiments, the composition comprises an emulsifier. In some embodiments, the emulsifier has a hydrophilic lipophilic balance (HLB) of at least about 10. Non-limiting examples of emulsifiers include polyoxyethylene hydrogenated castor oil, polyoxyethylene sorbitan fatty acid ester, polyoxyethylene castor oil, polyethylene glycol fatty acid ester, polyoxyethylene polyoxypropylene glycol, sucrose fatty acid ester, and lecithin. In some embodiments, the omega-3 fatty acids or derivatives thereof are present in an amount of about 50% to about 95% by weight of the total weight of the composition or by weight of the total fatty acids of the total composition. In some embodiments, the omega-3 fatty acid is EPA and/or DHA. In some embodiments, the EPA is present in amount at least about 95%, by weight, of all fatty acids present in the self-emulsifying composition. In some embodiments, the composition contains substantially no DHA. In yet some embodiments, the composition contains substantially no ethanol.

In some embodiments, the composition is a self-emulsifying composition comprising about 50% to about 95% by weight of the total weight of the composition with at least one compound selected from the group consisting of omega-3 polyunsaturated fatty acids and derivatives thereof (e.g., pharmaceutically acceptable salt and/or ester). In some embodiments, the composition comprises about 1% to about 20% by weight of the total weight of the composition, a sucrose fatty acid ester as an emulsifier having an HLB of at least about 10. In some embodiments, the composition comprises glycerin. In some embodiments, the composition comprises about 0% to about 5%, by weight of the total composition, ethanol. In some embodiments, the self-emulsifying composition comprises about 50% to about 95%, by weight of the total weight of the composition, at least one compound selected from the group consisting of omega-3 polyunsaturated fatty acids and derivatives thereof; about 1% to about 20%, by weight of the total weight of the composition, a sucrose fatty acid ester as an emulsifier having an HLB of at least about 10; glycerin; and about 0% to about 4%, by weight of the total weight of the composition, ethanol. In some embodiments, the sucrose fatty acid ester is one or more of: sucrose laurate, sucrose myristate, sucrose palmitate, sucrose stearate, or sucrose oleate. In some embodiments, the omega-3 polyunsaturated fatty acid is one or more of EPA, DHA, or derivatives thereof. In yet some embodiments, the omega-3 polyunsaturated fatty acid is ethyl-EPA and/or ethyl-DHA.

In some embodiments, the composition is a self-emulsifying composition comprising about 50% to about 95% by weight of the total weight of the composition, at least one compound selected from the group consisting of omega-3 polyunsaturated fatty acids and derivatives thereof (e.g., pharmaceutically acceptable salt and ester); and about 5% to about 50%, by weight, of the total weight of the composition an emulsifier having an HLB of at least about 10; wherein ethanol content is up to about 4% by weight of the total weight of the composition. In some embodiments, the omega-3 polyunsaturated fatty acid is EPA and/or DHA. In some embodiments, the composition does not contain ethanol. In some embodiments, the emulsifier is at least one member selected from the group consisting of polyoxyethylene hydrogenated castor oil, polyoxyethylene sorbitan fatty acid ester, polyoxyethylene castor oil, polyethylene glycol fatty acid ester, polyoxyethylene polyoxypropylene glycol, sucrose fatty acid ester, and lecithin. In some embodiments, the emulsifier is at least one member selected from the group consisting of polyoxyethylene hydrogenated castor oil, polyoxyethylene sorbitan fatty acid ester, polyoxyethylene castor oil, and sucrose fatty acid ester.

In some embodiments, the hydrogenated castor oil is at least one member selected from the group consisting of polyoxyethylene (20) hydrogenated castor oil, polyoxyethylene (40) hydrogenated castor oil, polyoxyethylene (50) hydrogenated castor oil, polyoxyethylene (60) hydrogenated castor oil, or polyoxyethylene (100) hydrogenated castor oil. In some embodiments, the polyoxyethylene sorbitan fatty acid ester is at least one member selected from the group consisting of polyoxyethylene sorbitan monooleate, polyoxyethylene sorbitan tristearate, polyoxyethylene sorbitan monostearate, polyoxyethylene sorbitan monopalmitate, and polyoxyethylene sorbitan monolaurate. In some embodiments, the sucrose fatty acid ester is at least one member selected from the group consisting of sucrose laurate, sucrose myristate, sucrose palmitate, sucrose stearate, and sucrose oleate.

In some embodiments, the composition contains a lecithin selected from the group consisting of soybean lecithin, enzymatically decomposed soybean lecithin, hydrogenated soybean lecithin, and egg yolk lecithin. In some embodiments, the composition contains a polyhydric alcohol, wherein the polyhydric alcohol is propylene glycol or glycerin. In some embodiments, the composition contains at least one member selected from the group consisting of EPA, DHA, and/or derivatives thereof (e.g., their pharmaceutically acceptable salt and ester), wherein the composition contains ethyl-EPA and/or ethyl-DHA. In some embodiments, the composition comprises an emulsifier having an HLB of at least about 10 and is about 10 to about 100 parts by weight in relation to 100 parts by weight of the at least one compound selected from the group consisting of omega-3 polyunsaturated fatty acids and/or derivatives thereof (e.g., pharmaceutically acceptable salt and/or ester).

In some embodiments, the self-emulsifying composition comprises about 70% to about 90%, by weight, eicosapentaenoic acid ethyl ester as a first medicinal component. In some embodiments, the composition further comprises about 0.5% to about 0.6%, by weight, water. In some embodiments, the composition comprises about 1% to about 29%, by weight, polyoxyethylene sorbitan fatty acid ester as an emulsifier. In some embodiments, the composition comprises about 1 part to about 25 parts, by weight, lecithin in relation to about 100 parts, by weight, eicosapentaenoic acid ethyl ester. In yet some embodiments, the composition comprises pitavastatin, rosuvastatin, or a salt thereof as a second medicinal component. In some embodiments, ethanol and/or polyhydric alcohol constitutes up to about 4% by weight of the total weight of the composition. In some embodiments, the composition comprises about 0.01 part to about 1 part, by weight, of pitavastatin or its salt in relation to about 100 parts, by weight, of the eicosapentaenoic acid ethyl ester, or about 0.03 part to about 5 parts, by weight, rosuvastatin or its salt in relation to about 100 parts, by weight, eicosapentaenoic acid ethyl ester as a second medicinal component. In some embodiments, the composition is encapsulated in a hard capsule and/or a soft capsule, wherein a capsule film of the soft capsule may contain gelatin. In some embodiments, the self-emulsifying composition further comprises polyoxyethylene hydrogenated castor oil and/or polyoxyethylene castor oil. In some embodiments, the emulsifier comprises polyoxyethylene sorbitan fatty acid ester and polyoxyethylene castor oil. In some embodiments, the pitavastatin, rosuvastatin, or a salt thereof is pitavastatin calcium or rosuvastatin calcium. In some embodiments, the lecithin is soybean lecithin. In some embodiments, the polyoxyethylene sorbitan fatty acid ester is polyoxyethylene (20) sorbitan monooleate.

In some embodiments, the self-emulsifying composition comprising E-EPA has improved bioavailability as compared to a standard E-EPA formulation. A standard E-EPA formulation is a formulation that is not self-emulsifying. In some embodiments, a self-emulsifying composition comprising about 1.8 g to about 3.8 g of E-EPA has substantially equivalent bioavailability to about 4 g E-EPA that is not formulated as a self-emulsifying composition. In some embodiments, the self-emulsifying composition comprising E-EPA is assessed for a bioequivalence to about 4 g E-EPA that is not formulated as a self-emulsifying composition using for example, U.S. Food and Drug Administration (FDA) guidelines.

In some embodiments, compositions useful in accordance with methods of the disclosure are orally deliverable. The terms “orally deliverable” or “oral administration” herein include any form of delivery of a therapeutic agent or a composition thereof to a subject wherein the agent or composition is placed in the mouth of the subject, whether or not the agent or composition is swallowed. Thus “oral administration” includes buccal and sublingual as well as esophageal administration. In one embodiment, the composition is present in a capsule, for example, a soft gelatin capsule.

In some embodiments, compositions useful in accordance with methods disclosed herein are administered via an enteral route. For example, compositions containing higher doses of EPA and/or derivatives thereof (e.g., 20 g) may be given by the enteric route (e.g., by tube feeding). In the ICU setting, critically ill patients with sepsis or ARDS may receive a dosage of EPA and/or derivatives thereof up to 20 g/day by the enteric route, or orally (e.g., by drinking the liquid).

The term “enteral route” of administration refers to the administration via any part of the gastrointestinal tract. Examples of enteral routes include oral, mucosal, buccal, and rectal route, or intragastric route.

A composition for use in accordance with the disclosure can be formulated as one or more dosage units. The terms “dose unit” and “dosage unit” herein refer to a portion of a pharmaceutical composition that contains an amount of a therapeutic agent suitable for a single administration to provide a therapeutic effect. Such dosage units may be administered once to a plurality (e.g., 1 to about 10, 1 to 8, 1 to 6, 1 to 4 or 1 to 2) of times per day, or as many times as needed to elicit a therapeutic response.

In one embodiment, compositions of the disclosure, upon storage in a closed container maintained at room temperature, refrigerated temperature (e.g., about 5 to about −10° C.), or frozen for a period of about 1, 2, 3, 4, 5, 6, 7, 8, 9, 10, 11, or 12 months, exhibit at least about 90%, at least about 95%, at least about 97.5%, or at least about 99% of the active ingredient(s) originally present therein.

### Therapeutic Methods

In some embodiments, the disclosure provides methods for treatment and/or prevention of endothelial dysfunction in a subject. Endothelial dysfunction can be a resulting condition of various cardiovascular risk factors and an independent predictor of cardiac or ischemic events. It usually precedes the development of atherosclerosis and is involved in lesion formation by the promotion of both the early and late mechanisms of atherosclerosis, including upregulation of adhesion molecules, increased chemokine secretion and leukocyte adherence, increased cell permeability, enhanced low-density lipoprotein oxidation, platelet activation, cytokine elaboration, and vascular smooth muscle cell proliferation and migration. The basic mechanisms involved in atherogenesis indicate that endothelial dysfunction represents a key early step in the development of atherosclerosis and is also involved in plaque progression and the occurrence of atherosclerotic complications. Endothelial dysfunction also includes diminished production or availability of nitric oxide and/or an imbalance in the relative contribution of endothelium-derived relaxing and contracting factors, which leads to an impairment of endothelium-dependent vasodilation. Additionally, endothelial dysfunction also comprises a specific state of endothelial activation characterized by a proinflammatory, proliferative, and procoagulatory states that favor progression to atherogenesis.

In some embodiments, the present disclosure provides methods of treating and/or preventing endothelial dysfunction in a subject, comprising administering to the subject a pharmaceutical composition comprising eicosapentaenoic acid and/or a derivative thereof. In some embodiments, the pharmaceutical composition is administered at an amount effective to provide a daily dose of about 1 g to about 20 g (e.g., about 10-20 g, about 1-12 g) of eicosapentaenoic acid and/or a derivative thereof to the subject.

In some embodiments, the present disclosure provides methods of increasing an activity of or an amount of heme oxygenase-1 (HO-1) in a subject via administration of the pharmaceutical compositions disclosed herein in any of their embodiments.

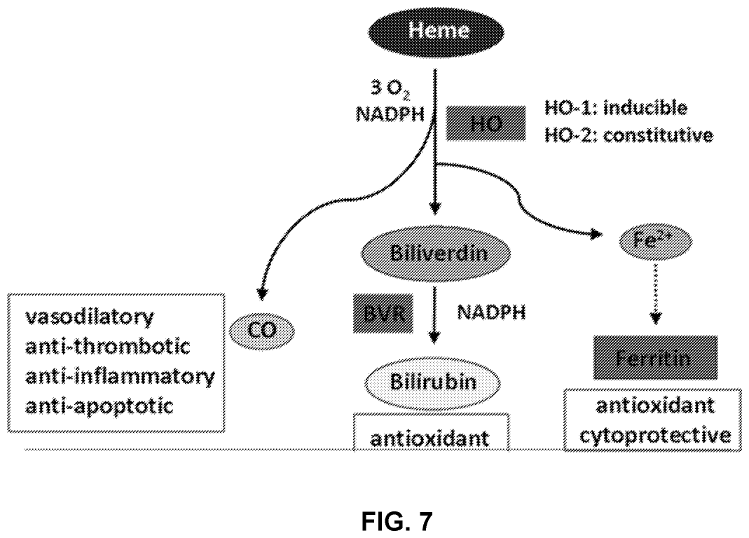

As illustrated in FIG. 7, heme oxygenase (HO) is an enzyme that catalyzes the degradation of heme thereby producing biliverdin (and bilirubin), ferrous ion, and carbon monoxide (CO) via a two-step process. In particular, heme oxygenase 1 (HMOX1, or HO-1), which is a member of the heat shock protein (HSP) family. HMOX1 may be a relevant target for diabetes, cardiovascular disease, hypertension, and pulmonary function.

Bilirubin represents a much larger cascade of antioxidant effects as only one response element of the larger heme oxidase pathway. The overall heme oxidase pathway yields at least three antioxidant metabolites, and also has vasodilatory, anti-thrombotic, anti-inflammatory, and anti-apoptotic effects.

In some embodiments, the present disclosure provides methods of activating antioxidant response elements (AREs) in a subject via administration of the pharmaceutical compositions disclosed herein in any of their embodiments. In some embodiments, the present disclosure provides methods of activating transcription factor Nrf2 in in a subject via administration of the pharmaceutical compositions disclosed herein in any of their embodiments.

Transcriptional activation of AREs may play a role in modulating oxidative stress and maintaining a redox balance. Transcription factors such as nuclear factor erythroid 2-related factor 2 (Nrf2) may be involved in this process. For example, Nrf2 may bind to the ARE and regulate the expression of several antioxidant genes in response to several oxidants and toxic stimuli. In particular, the trio of HMOX1, NQ01, and GST are antioxidants that are induced by the ARE, which may be induced by EPA activation of Nfr2 protein.

Based on a clinical study of human participants, the inventors have discovered that exposure to a high-dose ethyl EPA (i.e., eicosatetraenoic acid ethyl ester) is associated with fewer anemia AEs and higher hemoglobin and hematocrit. High-dose ethyl EPA exposure is also associated with higher levels of the heme derivative bilirubin, which is a potent antioxidant. As shown in FIG. 7, heme oxidase converts heme to biliverdin, and biliverdin reductase converts biliverdin to bilirubin. Both biliverdin and bilirubin are potent antioxidants, but bilirubin is likely the stronger of the two. Bilirubin can be converted back into biliverdin rather than being eliminated, thus perpetuating both compounds' antioxidant effects.

Studies where human umbilical vein endothelial cells (HUVEC) were exposed to EPA showed that EPA stimulates the anti-oxidant response element Nrf2 and in turn, heme oxygenase. Blocking heme oxygenase blocked EPA's ability to protect against H2O2-induced oxidative stress (see Lee S E, J Cardiovasc Pharm, 2015, incorporated herein by reference in its entirety). Heme oxygenase may protect against sepsis, HTN, atherosclerosis, acute lung injury, kidney injury, and pain. Accordingly, the cluster of increased hemoglobin and the antioxidant bilirubin could also be associated with other efficacy signals from the REDUCE-IT clinical trial, such as lower hypertensive adverse effects, lower Spontaneous Bacterial Peritonitis (SBP), lower proteinuria adverse effects, and lower microalbuminuria adverse effects.

Long-chain polyunsaturated fatty acids (e.g., EPA) and derivatives (e.g., oxidative metabolites such as oxylipins) may induce heme oxygenase such as HO-1. For example, EPA and oxylipins may activate Nrf2, which induces several antioxidant pathways including HO-1. As explained above, HO-1 inhibits inflammatory cascade, promotes pro-resolving cascade, and inhibits oxidative stress. HO-1 may mediate EPA or its derivatives inflammatory effects.

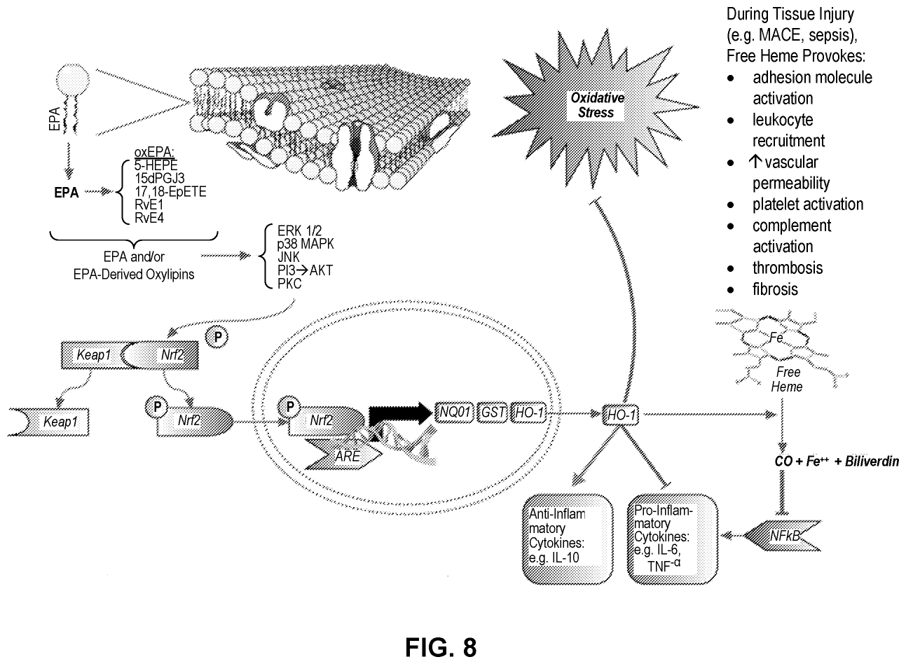

As shown in FIG. 8, linkages may exist among EPA, oxEPA (i.e. EPA-derived oxylipins/eicosanoids), Nrf2, and the AntiOxidant Response Element (ARE), leading to HO-1 production, and subsequent antioxidant and anti-inflammatory effects.

Specifically, EPA and oxylipins therefrom stimulate phosphorylation of Nrf2, resulting in its translocation to the nucleus where it interacts with the ARE, increasing expression of several antioxidant pathways, including heme oxygenase 1 (HO-1). The downstream antioxidant effects and anti-inflammatory effects are shown. The pathways to Nrf2 phosphorylation are probably tissue-specific, so several mediators are listed. For example, p38 MAPK is responsible for stimulating Nrf2 activation and HO-1 expression and activity when HUVEC cells are exposed to EPA.

Accordingly, the methods disclosed herein in any of their embodiments may exhibit vasodilatory, anti-thrombotic, anti-inflammatory, anti-apoptotic, antioxidant, and/or cytoprotective effects. The composition and/or methods disclosed herein may be useful for the treatment of diseases and/or disorders including, but not limited to, pneumonia, COPD, SIRS/sepsis/ARDS, acute lung injury, kidney injury, pain, atherosclerosis, hemolytic diseases (sickle-cell, thalassemia, hereditary spherocytosis), NAFLD/NASH, hematologic (red cell) disorders, platelet activation, fibrosis, pulmonary embolism, thrombosis, other thromboembolic diseases (e.g., venous thrombosis), MACE, DIC (disseminated intravascular coagulation), and other disorders involving excessive platelet activation. The composition and/or methods may be particularly useful for the treatment of diseases involving significant tissue injury, which raises levels of free heme to toxic levels. Furthermore, induction of HO-1 may prevent or treat complications in critically ill COVID-19 patients.

In some embodiments, the disclosure also provides methods for treatment and/or prevention of cardiovascular diseases or disorders in a subject. The term “cardiovascular-related disease and disorders” herein refers to any disease or disorder of the heart or blood vessels (i.e. arteries and veins) or any symptom thereof. Non-limiting examples of cardiovascular-related disease and disorders include hypertriglyceridemia, hypercholesterolemia, mixed dyslipidemia, coronary heart disease, vascular disease (e.g., vasculitis and pulmonary-renal syndromes), glomerular diseases (e.g., nephritis and nephropathy), thrombo-embolic diseases, stroke, atherosclerosis, arrhythmia, hypertension, myocardial infarction, and other cardiovascular events.

The term “treatment” in relation a given disease or disorder includes, but is not limited to, inhibiting the disease or disorder, for example, arresting the development of the disease or disorder; relieving the disease or disorder, for example, causing regression of the disease or disorder; or relieving a condition caused by or resulting from the disease or disorder, for example, relieving or treating symptoms of the disease or disorder. The term “prevention” in relation to a given disease or disorder means preventing the onset of disease or disorder development if none had occurred; preventing the disease or disorder from occurring in a subject that may be predisposed to the disease or disorder but has not yet been diagnosed as having the disease or disorder; and/or preventing further disease or disorder development if already present.

In some embodiments, the methods comprise administering to the subject about 1 g to about 20 g of EPA per day. For example, about 1 g, about 2 g, about 3 g, about 4 g, about 5 g, about 6 g, about 7 g, about 8 g, about 9 g, about 10 g, about 11 g, about 12 g, about 13 g, about 14 g, about 15 g, about 16 g, about 17 g, about 18 g, about 19 g, or about 20 g of EPA per day.

In some embodiments, the methods comprise administering a composition comprises EPA that is formulated such that when administered to the subject, the composition provides an amount of EPA effective to achieve an efficacy equivalent dose to about a 4 g dose of EPA but at a lower daily dose of EPA. In some embodiments, the lower daily dose of the EPA of is no more than about 3.8 g, no more than about 3.6 g, no more than about 3.4 g, no more than about 3.2 g, no more than about 3 g, no more than about 2.8 g, no more than about 2.6 g, or no more than about 2.5 g. In some embodiments, the lower daily dose of the EPA is reduced by at least about 10%, at least about 20%, at least about 30%, at least about 40% in the subject as compared to a baseline or placebo control. In one embodiment, administering the composition to the subject results in an improved pharmacokinetic profile in the subject as compared a control subject, wherein the subject and control subject are in either or fed or fasting state, and wherein the pharmacokinetic profile is defined by maximum serum concentration (Cₘₐₓ) and area under the curve (AUC). In some embodiments, the control subject is on a statin therapy and administered a placebo or other fatty acid composition such as Lovaza comprised of 365 mg of E-EPA and 375 mg of E-DHA. Similarly, in some embodiments, the methods comprise administering a composition comprises EPA that is formulated such that when administered to the subject, the composition provides an amount of EPA effective to achieve an efficacy equivalent dose to about a 10 g, 15 g, or 20 g dose of EPA but at a lower daily dose of EPA.

In some embodiments, the methods comprise administering to the subject the EPA for a period of time between about 3 days to about 1 year. In some embodiments, the subject is administered the EPA for about 3 days, about 4 days, about 5 days, about 6 days, about 1 week, about 1.5 weeks, about 2 weeks, about 2.5 weeks, about 3 weeks, about 1 month, about 2 months, about 3 months, about 4 months, about 5 months, about 6 months, about 7 months, about 8 months, about 9 months, about 10 months, about 11 months, or about 1 year.

In some embodiments, the subject has a fasting baseline triglyceride level of about 135 mg/dl to about 500 mg/dL, for example about 135 mg/dL to about 500 mg/dL, about 150 mg/dl to about 500 mg/dL, about 200 mg/dL to about 499 mg/dl or about 200 mg/dL to <500 mg/dL. In some embodiments, the subject has a fasting baseline triglyceride level of about 50 mg/dL to about 1500 mg/dL, for example about 50 mg/dl to about 1500 mg/dL, about 80 mg/dL to about 1500 mg/dL, about 50 mg/dL to about 190 mg/dl, about 80 mg/dL to about 190 mg/dl, about 190 mg/dl to about 250 mg/dL, about 250 mg/dl to about 1400 mg/dL. In one embodiment, the subject has a fasting baseline triglyceride level of about 80 mg/dL to about 1400 mg/dL. In some embodiments, the subject or subject group has a baseline triglyceride level (or median baseline triglyceride level in the case of a subject group), fed or fasting, of about 50 mg/dL, about 55 mg/dL, about 60 mg/dL, about 65 mg/dL, about 70 mg/dL, about 75 mg/dL, about 80 mg/dL, about 85 mg/dl, about 90 mg/dL, about 95 mg/dL, about 100 mg/dL, about 105 mg/dL, about 110 mg/dL, about 115 mg/dL, about 120 mg/dL, about 125 mg/dL, about 130 mg/dL, about 135 mg/dL, about 140 mg/dL, about 145 mg/dL, about 150 mg/dL, about 155 mg/dL, about 160 mg/dL, about 165 mg/dL, about 170 mg/dL, about 175 mg/dL, about 180 mg/dL, about 185 mg/dl, about 190 mg/dL, about 195 mg/dL, about 200 mg/dL, about 205 mg/dL, about 210 mg/dL, about 215 mg/dL, about 220 mg/dL, about 225 mg/dL, about 230 mg/dL, about 235 mg/dL, about 240 mg/dL, about 245 mg/dL, about 250 mg/dL, about 255 mg/dL, about 260 mg/dL, about 265 mg/dL, about 270 mg/dL, about 275 mg/dL, about 280 mg/dL, about 285 mg/dL, about 290 mg/dL, about 295 mg/dL, about 300 mg/dL, about 305 mg/dL, about 310 mg/dL, about 315 mg/dL, about 320 mg/dL, about 325 mg/dL, about 330 mg/dL, about 335 mg/dL, about 340 mg/dL, about 345 mg/dL, about 350 mg/dL, about 355 mg/dL, about 360 mg/dL, about 365 mg/dL, about 370 mg/dL, about 375 mg/dL, about 380 mg/dL, about 385 mg/dL, about 390 mg/dL, about 395 mg/dL, about 400 mg/dL, about 405 mg/dL, about 410 mg/dL, about 415 mg/dL, about 420 mg/dL, about 425 mg/dL, about 430 mg/dL, about 435 mg/dL, about 440 mg/dL, about 445 mg/dL, about 450 mg/dL, about 455 mg/dL, about 460 mg/dL, about 465 mg/dL, about 470 mg/dL, about 475 mg/dL, about 480 mg/dL, about 485 mg/dL, about 490 mg/dL, about 495 mg/dL, about 500 mg/dL, about 1000 mg/dL, about 1100 mg/dl, about 1200 mg/dL, about 1300 mg/dL, about 1400 mg/dL, about 1500 mg/dL, about 2000 mg/dL, about 2500 mg/dL, about 3000 mg/dL, about 3500 mg/dL, about 4000 mg/dL, about 4500 mg/dL, about 5000 mg/dl, or greater than about 5000 mg/dL. In some embodiments, the subject or subject group has a baseline triglyceride level (or median baseline triglyceride level in the case of a subject group), fed or fasting, greater than or equal to 80 mg/dl, greater than or equal to about 100 mg/dL, greater than or equal to about 120 mg/dl greater than or equal to about 150 mg/dL, greater than or equal to about 175 mg/dL, greater than or equal to about 250 mg/dL, or greater than equal to about 500 mg/dL, for example about 190 mg/dl to about 250 mg/dL, about 80 mg/dl to about 190 mg/dL, about 250 mg/dL to about 1400 mg/dL, about 200 mg/dl to about 500 mg/dL, about 300 mg/dL to about 1800 mg/dL, about 500 mg/dl to about 1500 mg/dL, or about 80 mg/dL to about 1500 mg/dL.

In some embodiments, the subject or subject group is also on stable therapy with a statin (with or without ezetimibe). In some embodiments, the subject or subject group also has established cardiovascular disease or is at high risk for establishing cardiovascular disease. In some embodiments, the subject's statin therapy includes administration of one or more statins. For example, and without limitation, the subject's statin therapy may include one or more of: atorvastatin, fluvastatin, lovastatin, pitavastatin, pravastatin, rosuvastatin, and simvastatin. In some embodiments, the subject is additionally administered one or more of: amlodipine, ezetimibe, niacin, and sitagliptin. In some embodiments, the subject's statin therapy includes administration of a statin and ezetimibe. In some embodiments, the subject's statin therapy includes administration of a statin without ezetimibe.

In some embodiments, the statin therapy is classified as monotherapies, combinations, and or 3-hydroxy-3-methyl-glutaryl-coenzyme A (HMG CoA) reductase inhibitor combinations. In some embodiments, the monotherapies include simvastatin, lovastatin, pravastatin, fluvastatin, atorvastatin, cerivastatin, rosuvastatin, or pitavastatin. In some embodiments, the combinations include lovastatin and nicotinic acid, simvastatin and ezetimibe, pravastatin and fenofibrate, simvastatin and fenofibrate, atorvastatin and ezetimibe, or rosuvastatin and ezetimibe. In some embodiments, the HMG CoA inhibitor combinations include simvastatin and acetylsalicylic acid; pravastatin and acetylsalicylic acid; atorvastatin and amlodipine; simvastatin, acetylsalicylic acid, and ramipril; rosuvastatin and acetylsalicylic acid; atorvastatin, acetylsalicylic acid, and ramipril; rosuvastatin, amlodipine, and lisinopril; atorvastatin and acetylsalicylic acid; rosuvastatin and amlodipine; rosuvastatin and valsartan; atorvastatin, amlodipine, and perindopril; atorvastatin, acetylsalicylic acid, and perindopril; rosuvastatin, perindopril, and indapamide; rosuvastatin, amlodipine, and perindopril; or atorvastatin and perindopril.

In some embodiments, the statin therapy is a low, medium (i.e., moderate), or high intensity statin therapy. In some embodiments, the low intensity statin therapy includes about 5 mg to about 10 mg of simvastatin. In some embodiments, the medium intensity statin therapy includes about 5 mg to about 10 mg of rosuvastatin, about 10 mg to about 20 mg of atorvastatin, about 20 mg to about 40 mg of simvastatin, or about 10 mg to about 20 mg of simvastatin plus about 5 mg to about 10 mg of ezetimibe. In some embodiments, the high intensity statin therapy includes about 20 mg to about 40 mg rosuvastatin, about 40 mg to about 80 mg of atorvastatin, about 80 mg of simvastatin, or about 40 mg to about 80 mg of simvastatin plus about 5 mg to about 10 mg of ezetimibe.

In some embodiments, the subject's statin therapy does not include administration of 200 mg or more per day of niacin and/or fibrates. In some embodiments, the subject is not on concomitant omega-3 fatty acid therapy (e.g., is not being administered or co-administered a prescription and/or an over-the-counter composition comprising an omega-3 fatty acid active agent). In some embodiments, the subject is not administered or does not ingest a dietary supplement comprising an omega-3 fatty acid.

In some embodiments, the subject has established cardiovascular (CV) disease (“CV disease” or “CVD”). The status of a subject as having CV disease can be determined by any suitable method known to those skilled in the art. In some embodiments, a subject is identified as having established CV disease by the presence of any one of: documented coronary artery disease, documented cerebrovascular disease, documented carotid disease, documented peripheral arterial disease, or combinations thereof. In some embodiments, a subject is identified as having CV disease if the subject is at least 45 years old and: (a) has one or more stenosis of greater than 50% in two major epicardial coronary arteries; (b) has had a documented prior MI; (c) has been hospitalized for high-risk NSTE ACS with objective evidence of ischemia (e.g., ST-segment deviation and/or biomarker positivity); (d) has a documented prior ischemic stroke; (e) has symptomatic artery disease with at least 50% carotid arterial stenosis; (f) has asymptomatic carotid artery disease with at least 70% carotid arterial stenosis per angiography or duplex ultrasound; (g) has an ankle-brachial index (“ABI”) of less than 0.9 with symptoms of intermittent claudication; and/or (h) has a history of aorto-iliac or peripheral arterial intervention (catheter-based or surgical).

In some embodiments, the subject or subject group being treated in accordance with methods of the disclosure has a high risk for developing CV disease. For example and without limitation, a subject or subject group has a high risk for developing CV disease if the subject or subject in a subject group is age about 50 or older, has diabetes mellitus (Type 1 or Type 2), and at least one of: (a) is a male age about 55 or older or a female age about 65 or older; (b) is a cigarette smoker or was a cigarette smoker who stopped less than about 3 months prior; (c) has hypertension (e.g., a blood pressure of about 140 mmHg systolic or higher, or greater than about 90 mmHg diastolic); (d) has an HDL-C level of less than or equal to about 40 mg/dL for men or less than or equal to about 50 mg/dL for women; (e) has an hs-CRP level of greater than about 3.0 mg/L; (f) has renal dysfunction (e.g., a creatinine clearance (“CrCL”) of greater than about 30 mL/min and less than about 60 mL/min); (g) has retinopathy (e.g., defined as any of: non-proliferative retinopathy, pre-proliferative retinopathy, proliferative retinopathy, maculopathy, advanced diabetic eye disease, or history of photocoagulation); (h) has microalbuminuria (e.g., a positive micral or other strip test, an albumin/creatinine ratio of greater than or equal to about 2.5 mg/mmol, or an albumin excretion rate on timed collection of greater than or equal to about 20 mg/min all on at least two successive occasions); (i) has macroalbuminuria (e.g., Albustix or other dip stick evidence of gross proteinuria, an albumin/creatinine ratio of greater than or equal to about 25 mg/mmol, or an albumin excretion rate on timed collection of greater than or equal to about 200 mg/min all on at least two successive occasions); and/or (j) has an ankle-brachial index of less than about 0.9 without symptoms of intermittent claudication.

In some embodiments, the subject's baseline lipid profile is measured or determined prior to administering the composition to the subject. Lipid profile characteristics can be determined by any suitable method known to those skilled in the art including, for example, by testing a fasting or non-fasting blood sample obtained from the subject using standard blood lipid profile assays. In some embodiments, the subject has one or more of: a baseline non-HDL-C value of about 200 mg/dl to about 300 mg/dL; a baseline total cholesterol value of about 250 mg/dl to about 300 mg/dL; a baseline VLDL-C value of about 140 mg/dl to about 200 mg/dL; a baseline HDL-C value of about 10 mg/dl to about 30 mg/dL; a baseline LDL-C value of about 40 mg/dL to about 100 mg/dL; and/or a baseline hs-CRP level of about 2 mg/dl or less.

In some embodiments, the cardiovascular event for which risk is reduced is one or more of: cardiovascular death; nonfatal myocardial infarction; nonfatal stroke; coronary revascularization; unstable angina (e.g., unstable angina determined to be caused by myocardial ischemia by, for example, invasive or non-invasive testing, and requiring hospitalization); cardiac arrest; peripheral cardiovascular disease requiring intervention, angioplasty, bypass surgery, or aneurysm repair; death; sudden cardiac death, sudden death, and onset of new congestive heart failure. In some embodiments, the cardiovascular event is a first, second, third, fourth, or more cardiovascular event experienced by the subject.

In some embodiments, the subject is administered about 1 g to about 20 g of the composition per day for about 4 months, about 1 year, about 1.25 years, about 1.5 years, about 1.75 years, about 2 years, about 2.25 years, about 2.5 years, about 2.75 years, about 3 years, about 3.25 years, about 3.5 years, about 3.75 years, about 4 years, about 4.25 years, about 4.5 years, about 4.75 years, about 5 years, or more than about 5 years. Thereafter, in some embodiments the subject exhibits one or more of:
(a) a reduction in triglyceride levels compared to baseline or control;
(b) a reduction in Apolipoprotein B (Apo B) levels compared to baseline or control;
(c) an increase in high-density lipoprotein cholesterol (HDL-C) levels compared to baseline or control;
(d) no increase or increase in low-density lipoprotein cholesterol (LDL-C) levels compared to baseline or control;
(e) a reduction in LDL-C levels compared to baseline;
(f) a reduction in non-HDL-C levels compared to baseline or control;
(g) an increase in non-HDL-C levels compared to baseline or control;
(h) a reduction in very low-density lipoprotein cholesterol (VLDL-C) levels compared to baseline or control;
(i) a reduction in total cholesterol levels compared to baseline or control;
(j) a reduction in hs-CRP levels compared to baseline or control;
(k) a reduction in platelet aggregation as compared to baseline or control;
(l) a reduction in inflammation as compared to baseline or control;
(m) an increase in nitric oxide (NO) bioavailability as compared to baseline or control;
(n) an increase in endothelial function as compared to baseline or control;
(o) a reduction in a risk for thrombosis as compared to baseline or control;
(p) a reduction in a risk for atherosclerosis as compared to baseline or control; and
(q) a reduction in a risk for cardiovascular diseases or disorders as compared to baseline or control.

In one embodiment, methods of the present disclosure comprise measuring baseline levels of one or more markers set forth in (a)-(q) above prior to dosing the subject or subject group. In some embodiments, the methods comprise administering a composition as disclosed herein to the subject after baseline levels of one or more markers set forth in (a)-(q) are determined, and subsequently taking an additional measurement of said one or more markers.

In some embodiments, upon treatment with a composition of the present disclosure, the subject exhibits one or more of:
(a) a reduction in triglyceride levels of at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, or at least about 75% as compared to baseline or control;
(b) a reduction in Apo B levels of at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, or at least about 75% as compared to baseline or control;
(c) an increase in HDL-C levels of at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, or at least about 75% as compared to baseline or control;
(d) no increase or an increase in LDL-C levels of less than 30%, less than 20%, less than 10%, less than 5% as compared to baseline or control;
(e) a reduction in LDL-C levels of at least about 5%, at least about 10%, at least about 15%, at least about 20%, at least about 25%, at least about 30%, at least about 35%, at least about 40%, at least about 45%, at least about 50%, or at least about 55% as compared to baseline or control;
(f) a reduction in non-HDL-C levels of at least about 1%, at least about 3%, at least about 5%, at least about 10%, at least about 15%, at least about 20%, at least about 25%, at least about 30%, at least about 35%, at least about 40%, at least about 45%, or at least about 50% as compared to baseline or control;
(g) an increase in non-HDL-C levels of less than 30%, less than 20%, less than 10%, less than 5% (actual % change or median % change), or no increase in non-HDL-C levels as compared to baseline or control;
(h) a reduction in VLDL-C levels of at least about 5%, at least about 10%, at least about 15%, at least about 20%, at least about 25%, at least about 30%, at least about 35%, at least about 40%, at least about 45%, at least about 50%, or at least about 100% compared to baseline or control;
(i) a reduction in total cholesterol levels of at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, or at least about 75% as compared to baseline or control; and/or
(j) a reduction in hs-CRP levels of at least about 5%, at least about 10%, at least about 15%, at least about 20%, at least about 25%, at least about 30%, at least about 35%, at least about 40%, at least about 45%, at least about 50%, or at least about 100% as compared to baseline or control;
(k) a reduction in platelet aggregation of at least about 5%, at least about 10%, at least about 15%, at least about 20%, at least about 25%, at least about 30%, at least about 35%, at least about 40%, at least about 45%, at least about 50%, or at least about 100% as compared to baseline or control;
(l) a reduction in inflammation of at least about 5%, at least about 10%, at least about 15%, at least about 20%, at least about 25%, at least about 30%, at least about 35%, at least about 40%, at least about 45%, at least about 50%, or at least about 100% as compared to baseline or control;
(m) an increase of NO bioavailability of at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 100% as compared to baseline or control;
(n) an increase of endothelial function of at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 100% as compared to baseline or control;
(o) a reduction in a risk for thrombosis of at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 100% as compared to baseline or control;
(p) a reduction in a risk for atherosclerosis of at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 100% as compared to baseline or control;
(q) a reduction in a risk for cardiovascular diseases and disorders of at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 100% as compared to baseline or control;
(r) an increase in the activity of HO-1 of at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 100% as compared to baseline or control;
(s) an increase in the amount of HO-1 of at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 100% as compared to baseline or control; and
(t) an increase in the activity of transcription factor Nrf2 of at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 100% as compared to baseline or control.

The present disclosure further relates to a diagnostic kit or diagnostic method based on a biomarker. In some embodiments, the diagnostic method involves measuring a concentration of a biomarker before and/or after the composition disclosed herein in any of the embodiments is administered. As used herein, the term “biomarker” refers to a characteristic that is objectively measured and evaluated as an indicator of normal biological processes, pathogenic processes or pharmacological responses to a therapeutic intervention. Exemplary applicable biomarkers include, without limitation, HMOX1 (HO-1), HMOX2 (HO-2), NFE2L2, COX2, NQ01, GST, and ACE (in lung tissue). In some embodiments, the biomarker is HO-1.

The concentration of the biomarker (e.g., HO-1) may be measured with an antibody-based method (e.g. an ELISA, radioimmunoassay (RIA)) or a nitric oxide assay (also known as Griess reagent assay). The protocol for measuring the concentration of the biomarker is known to those of ordinary skill, for example by performing the steps outlined in the commercially available assay kit sold by Sigma-Aldrich, Thermo Fisher Scientific, R & D Systems, ZeptoMetrix Inc., Cayman Inc., Abcam, Trevigen, Dojindo Molecular Technologies, Biovision, and Enzo Life Sciences.

As used herein, the term “antibody-based method” refers to any method with the use of an antibody including, but not limited to, enzyme-linked immunosorbent assay (ELISA), Western blotting, immunoprecipitation (IP), enzyme linked immunospot (ELISPOT), immunostaining, immunohistochemistry, immunocytochemistry, affinity chromatography, and the like.

In some embodiments, an ELISA is used. The term ELISA refers to a method of detecting the presence and concentration of a biomarker in a sample. There are several variants of ELISA, including, but not limited to, sandwich ELISA, competitive ELISA, indirect ELISA, ELISA reverse, and the like. The ELISA assay may be a singleplex assay or a multiplex assay, which refers to a type of assay that simultaneously measures multiple analytes in a single run/cycle of the assay.

The term “sample” includes any biological sample taken from the subject including a cell, tissue sample, or bodily fluid. For example, a sample may include a skin sample, a cheek cell sample, saliva, or blood cells. A sample can include, without limitation, a single cell, multiple cells, fragments of cells, an aliquot of a body fluid, whole blood, platelets, serum, plasma, red blood cells, white blood cells, endothelial cells, tissue biopsies, synovial fluid, and lymphatic fluid.

In some embodiments, the present disclosure provides a diagnostic method of assessing a suitability, dosage, and/or duration of the method disclosed previously (i.e., treating or preventing endothelial dysfunction, increasing an activity of or an amount of heme oxygenase-1 (HO-1), and/or activating antioxidant response elements (AREs) in a subject, via administering to the subject a pharmaceutical composition comprising eicosapentaenoic acid (EPA) and/or a derivative thereof, and optionally an additional polyunsaturated fatty acid or a derivative thereof which is chemically distinct from the EPA or the derivative thereof such as an oxylipin. The diagnostic method includes, prior to the administration, determining a concentration of HO-1 and/or a nucleotide sequence encoding HO-1, in bodily fluid or non-neural tissue obtained from the subject, and comparing the concentration with a corresponding concentration of HO-1 and/or an HO-1 encoding nucleotide sequence in a corresponding bodily fluid or non-neural tissue obtained from at least one control person, wherein a reduced concentration is used to determine the suitability, dosage, and/or duration.

In some embodiments, the bodily fluid is selected from plasma and cerebrospinal fluid and the tissue is selected from lymphocytes and fibroblasts, or the concentration of mRNA is determined and the tissue is selected from lymphocytes and fibroblasts.

As used herein, the control person may be a person who lacks a disease trait of interest. For instance, for sickle cell disease, a negative control person would be someone who lacks hemolytic disease for both chronic and acute-crisis. On the other hand, a positive control person would be someone with chronic hemolytic disease that doesn't have acute-crises (e.g. spherocytosis, thalassemia).

Traditionally, indications for EPA have been predicated on having clinical risk factors (very high TG or CV risk factors). But as an agent to reap health benefits of robust HO-1 activation, the diagnostic could be used to identify deficiency states relative the level appropriate to a given disease/indication. Thus, the control person can be identified by the level of HO-1. In some embodiments, the disease population of interest may be classified as having a HO-1 level that is lower than the 5th to the 25th population percentile, lower than the 10th to the 20th population percentile, or lower than about the 15th population percentile. However, the percentiles could be further adjusted based on age, race, and/or gender of the subject.

In some embodiments, when the concentration of the biomarker (e.g., HO-1) has not returned to the normal concentration, the method further comprises increasing the dose of the pharmaceutical composition (e.g., EPA, the derivative of EPA, and optionally optionally an additional polyunsaturated fatty acid or a derivative thereof which is chemically distinct from the EPA or the derivative thereof such as an oxylipin, or the combination thereof) by at least 5%, at least 10%, or at least 30%, up to 50%, up to 60%, or up to 80% of an initial dose which would be based on physicians' evolving knowledge and the general skill in the art. The subject may be administered with the increased dosage for a longer period (e.g. more than 3 days, more than 2 weeks, or more than 1 year) than the duration with the initial dose. As used herein, the term “normal concentration” refers to a concentration of the biomarker identified in a normal healthy subject (e.g., the control person).

In some embodiments, the administration is stopped once the subject is treated.

In some embodiments, any of the methods disclosed herein are used in treatment or prevention of a subject or subjects that consume a traditional Western diet. In one embodiment, the methods of the disclosure include a step of identifying a subject as a Western diet consumer or prudent diet consumer and then treating the subject if the subject is deemed a Western diet consumer. The term “Western diet” herein refers generally to a typical diet consisting of, by percentage of total calories, about 45% to about 50% carbohydrate, about 35% to about 40% fat, and about 10% to about 15% protein. A Western diet may alternately or additionally be characterized by relatively high intakes of red and processed meats, sweets, refined grains, and desserts, for example, where more than 50%, more than 60%, or more or 70% of total calories come from these sources.

In some embodiments, a composition as described herein is administered to a subject once or twice per day. In some embodiments, 1, 2, 3, or 4 capsules, each containing about 1 g of a composition as described herein, are administered to a subject daily. In some embodiments, 1 or 2 capsules, each containing about 1 g of a composition as described herein, are administered to the subject in the morning, for example, between about 5 am and about 11 am, and 1 or 2 capsules, each containing about 1 g of a composition as described herein, are administered to the subject in the evening, for example between about 5 pm and about 11 pm.

### EXAMPLES

### Example 1: Eicosapentaenoic Acid Reduces Cytokine-Induced Expression of Multiple Proteins Related to Platelet Activation and Aggregation in Pulmonary and Vascular Endothelium

Platelet aggregation and thrombus formation are linked to cardiovascular (CV) disease and its clinical manifestations. Eicosapentaenoic acid (EPA), an omega-3 fatty acid, reduced CV events, including ischemic stroke, in high-risk patients (see the REDUCE-IT clinical trial, NCT01492361). Here, the effects of EPA were examined on expression of proteins involved in platelet activation and aggregation in both pulmonary and vascular endothelial cells (ECs) under conditions of inflammation with cytokine challenge.

Human pulmonary ECs (PECs) and umbilical vein ECs (HUVECs) were treated with vehicle or EPA (40/10 μM in PECs/HUVECs) and IL-6 (12 ng/ml) for 24 hours. Proteomic analysis was performed using liquid chromatography/mass spectroscopy. Proteins which showed significantly (p<0.05) greater than 1-fold change between treatment groups were analyzed.

EPA significantly downregulated a total of 36 proteins involved in platelet activation, signaling, aggregation, in EC following IL-6 exposure. Platelet endothelial cell adhesion molecule (PECAM) was the only common protein that was significantly downregulated by EPA in both HUVECs and PECs (1.2-fold). In PECs, EPA significantly modulated 26 other proteins related to platelet activation, including amyloid-beta precursor protein (1.1-fold decrease) and thrombin receptor (1.3-fold decrease), while in HUVECs there were nine other proteins modulated related to platelet activation, including superoxide dismutase (1.6-fold increase).

EPA significantly reduced expression of PECAM in ECs from different tissues, as well as other proteins associated with platelet activity. These findings suggest a potentially novel antithrombotic effect for EPA that may contribute to reduced ischemic events.

### Example 2: Eicosapentaenoic Acid Modulates Endothelial Function and Inflammatory Protein Expression from Pulmonary and Vascular Tissues Following Cytokine Challenge

Endothelial cell (EC) dysfunction is characterized by reduced nitric oxide (NO) bioavailability and contributes to inflammation and atherosclerosis. EPA reduced cardiovascular (CV) events in high-risk patients (REDUCE-IT, NCT01492361), but the mechanisms are not fully understood. Therefore, the effects of EPA were evaluated on expression of proteins and NO bioavailability in vascular and pulmonary ECs under conditions of inflammation.

Human pulmonary ECs (PECs) and umbilical vein ECs (HUVECs) were treated with vehicle or EPA (40/10 μM in PECs/HUVECs) and IL-6 (12 ng/ml) for 24 hours. Proteomic analysis was performed using liquid chromatography/mass spectroscopy. Protein expression significantly (p<0.05) greater than 1-fold change between treatment groups was analyzed. NO release was measured in HUVECs using tandem porphyrinic nanosensors following stimulation with calcium.

EPA significantly modulated more than 50 identical proteins in pulmonary and vascular ECs following IL-6 exposure. These included seven proteins related to neutrophil degranulation and cytokine signaling, including integrin alpha-V (1.5/1.1-fold decrease in HUVECs/PECs), and five proteins linked to EC function and inflammation, including heme-oxygenase-1 (1.6/1.9-fold increase in HUVECs/PECs). In HUVECs, treatment with EPA also significantly increased NO release by 13% (p<0.05) relative to IL-6 alone.

EPA favorably modulated expression of EC proteins associated with inflammation and improved NO bioavailability during IL-6 exposure. These studies support favorable anti-inflammatory effects of EPA on ECs in multiple vascular beds that may contribute to reduced CV risk.

### Example 3: Eicosapentaenoic Acid Increases Omega-3 Fatty Acid Content and Reduces Inflammatory Protein Levels in Pulmonary Endothelial Cells During IL-6 Exposure

During inflammation, pulmonary endothelial cells (PECs) release vasoconstrictive and pro-inflammatory mediators that lead to vascular and pulmonary hypertension. The omega-3 fatty acid (n3FA) EPA has vascular benefits that contribute to reduced events in patients with cardiovascular (CV) risk (REDUCE-IT, NCT01492361). The effects of EPA on fatty acid content and protein expression in PECs were evaluated under conditions of inflammation.

Human PECs were pretreated with vehicle or EPA (40 μM) for 2 hours, then challenged with IL-6 at 12 ng/ml for 24 hours. Proteomic analysis was performed using LC/MS to capture relative expression levels. Only significant changes in protein expression between treatment groups >2-fold were analyzed. Levels of intercellular adhesion molecule-1 (ICAM-1) were measured by immunochemistry. EPA, docosahexaenoic acid (DHA), docosapentaenoic acid (DPA), palmitic acid (PA), linoleic acid (LA) and arachidonic acid (AA) content was measured by GC/MS.

PECs pretreated with EPA and challenged with IL-6 down-regulated more than 60 proteins. EPA significantly downregulated angiotensin converting enzyme (ACE) and ICAM-1 compared to IL-6 (p<0.05). These changes correlated with increases in EPA (0.37±0.03 mg/g protein), DPA (0.25±0.07 mg/g protein) and DHA (0.023±0.014 mg/g protein) levels compared to IL-6 treatment alone (below detection limit). EPA also significantly reduced levels of PA (34% p<0.05), while levels of LA and AA did not change.

EPA significantly reduced expression of proteins associated with vasoconstriction and inflammation that correlated with increased n3FAs and lower PA. These findings indicate a novel benefit of EPA that may have implications for vascular and pulmonary function.

### Example 4: Eicosapentaenoic Acid Reduced Levels of Angiotensin Converting Enzyme and Caveolin-1 in Pulmonary Endothelial Cells Following Cytokine Treatment

Pulmonary endothelial cell (EC) dysfunction leads nitric oxide synthase (NOS) uncoupling and release of vasoconstrictors like angiotensin II and endothelin-1. The omega-3 fatty acid (n3FA) EPA reduced cardiovascular (CV) events in high-risk patients (REDUCE-IT, NCT01492361), but the mechanism is not understood. The effects of EPA were tested on expression of proteins that modulate NOS, including caveolin-1 and heat shock protein-90 (Hsp90), and proteins that effect vasoconstriction, including endothelin-converting enzyme-1 (ECE-1) and angiotensin converting enzyme (ACE).

Human pulmonary ECs were treated with vehicle or EPA for 24 h after exposure to IL-6 at 12 ng/ml for 2 hours. Proteomic analysis was performed using liquid chromatography/mass spectroscopy to capture relative expression levels. Only significant changes in protein expression between treatment groups >1-fold with a p-value less than 0.05 were analyzed.

Pulmonary ECs treated with EPA following IL-6 exposure showed significant changes in expression of more than 400 proteins including those that mediate inflammation and vasodilation. EPA significantly downregulated caveolin-1 (1.3-fold), an inhibitor on NOS, and increased levels of Hsp90 (1.1-fold) compared to IL-6 (p<0.05). EPA significantly reduced expression of additional proteins linked to NOS inhibition and as well as ACE and ECE-1 levels (1.1-fold, 1.3-fold, respectively p<0.05).

EPA favorably modulated expression of proteins associated with NOS activation and vasoconstriction following IL-6 exposure, including ACE and ECE-1. These studies support a novel effect of EPA on pulmonary ECs that may contribute to reduced CV disease progression.

### Example 5: Eicosapentaenoic Acid (EPA) Increases Heme Oxygenase-1 Expression in Endothelial Cells Under Conditions of Inflammation Unlike Docosahexaenoic Acid (DHA)

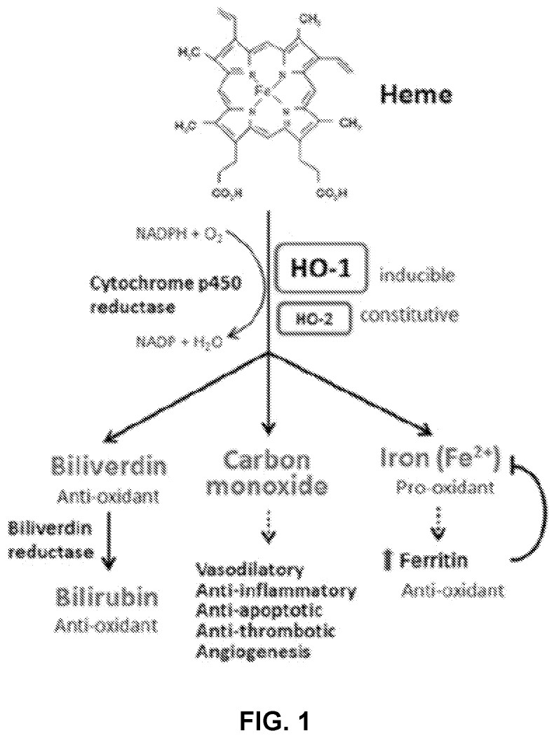

The inducible isoform of heme oxygenase, heme oxygenase-1 (HMOX-1), is a cytoprotective enzyme whose primary function is the catabolism of heme. The byproducts of the enzymatic break down of heme are free iron (Fe²⁺), carbon monoxide (CO), and biliverdin, which is converted to bilirubin by biliverdin reductase (FIG. 1). Together, bilirubin, biliverdin, CO, and Fe²⁺-induced upregulation of ferritin are believed to contribute to the anti-inflammatory actions of HMOX-1. Therapeutic agents that enhance HMOX-1 expression or its products are expected to have cardiovascular (CV) benefits as evidenced in models of atherosclerosis. The present study compared the effects of EPA versus DHA on global protein expression, including heme oxygenase-1, and correlated with nitric oxide release from vascular ECs under inflammatory conditions with the cytokine IL-6.

Primary human umbilical vein endothelial cells (HUVECs) were purchased from Lonza (Manassas, VA). Cells were cultured in the recommended complete endothelial cell growth medium and maintained at 37° C. in a 95% air/5% CO2 humidified incubator. As recommended by the supplier, cells were supplied with fresh medium every other day and propagated by an enzymatic (trypsin) procedure as previously described. Cell culture medium also contained 2% FBS, which contained albumin to facilitate efficient delivery of fatty acid treatment. The fatty acids EPA and DHA were purchased from Sigma-Aldrich (Saint Louis, MO) and solubilized in redistilled ethanol under nitrogen atmosphere. The various acid stock solutions were stored at −20° C. until use.

HUVECs were treated with vehicle, EPA, or DHA (10 μM) for 2 hours, and then challenged with IL-6 (12 ng/ml) for 24 h hours. After incubation, cells were pelleted and frozen at −80° C. until proteomic analysis was performed. Cell pellets were lysed using methanol/chloroform extraction. Proteins were denatured, reduced, alkylated, and trypsin digested. Samples were then prepared for Tandem Mass Tag (TMT) 10plex labeling. A bicinchoninic acid (BCA) assay was performed on each sample to quantify the total protein in each sample, which is important to confirm equal amounts of each sample are added to the multiplex sample. Each peptide in a sample was given a unique, low molecular weight (typically 126-130 Da), and then the samples were combined. Relative protein expression levels among the various treatments were measured using LC/MS proteomic techniques. Following protein digest, peptides are separated over a reverse phase column and then identified based on their mass.

Each multiplexed sample was then fractionated to increase the overall protein coverage using high pH reversed phase fractionation and analyzed by LC/MS using a Dionex UltiMate 3000 RSLC in tandem with a Q-Exactive/Lumos Orbitrap Mass Spectrometer. The chromatography was performed using a 2-hour gradient on a Thermo Pepmap C18 column (100 Å pore size, 3.0 μm particle size, 100 μM×150 mm) set at 50° C. Mobile phase A was water with 0.1% formic acid, and mobile phase B was acetonitrile with 0.1% formic acid. Proteins that showed a fold change >1.0 and p<0.05 for the relevant comparisons were considered significant and further analyzed.

NO release from HUVECs was measured as previously described. Briefly, in parallel with the cellular preparations for proteomic analysis, HUVECs were stimulated with calcium ionophore and NO was measured using tandem porphyrinic nanosensors.

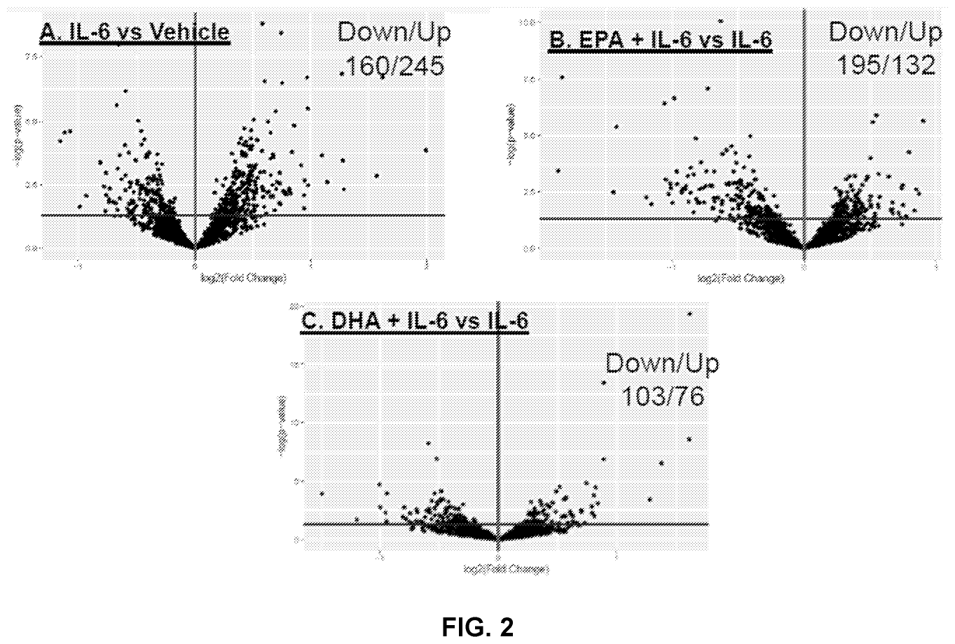

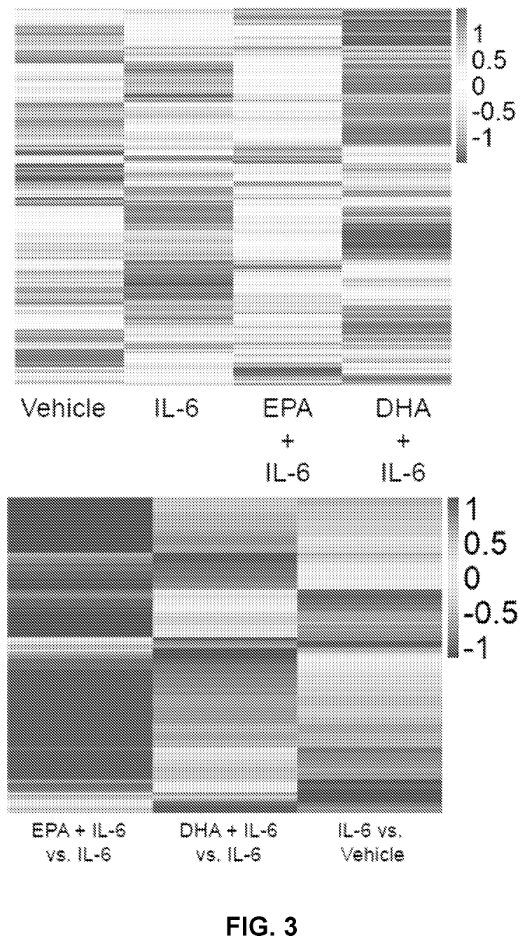

As shown in FIGS. 2-3, cells pretreated with EPA and DHA significantly down/up-regulated expression of 195/132 and 103/76 proteins, respectively, compared with IL-6 alone. However, as shown in Tables 1 and 2 below, only EPA upregulated inducible HMOX-1 by 160% (p=0.02), and only EPA significantly increased NO release by 13% (p=0.04) compared to IL-6 alone.

TABLE 1
Effects of EPA versus DHA on HMOX-1
Expression Relative to IL-6

| Treatment | Fold Change Increase | P value |
|---|---|---|
| EPA + IL-6 vs. IL-6 | 1.58 | 0.021 |
| DHA + IL-6 vs. IL-6 | — | — |

TABLE 2
Effects of EPA versus DHA on Nitric Oxide
Release from HUVECs Changes with IL-6 (values
are mean ± SEM; N = 4-5)

| Treatment | NO Release (nM) | Significance (vs. IL-6) |
|---|---|---|
| Vehicle | 468 ± 15 | p < 0.01 |
| IL-6 | 371 ± 14 | — |
| EPA + IL-6 | 421 ± 12 | p < 0.05 |
| DHA + IL-6 | 404 ± 15 | n.s. |

Thus, these data show that EPA significantly increased expression of HMOX-1 and NO release under conditions of inflammation. These beneficial effects of EPA were not reproduced by DHA and may contribute to preserved vascular EC function and reduced CV risk as demonstrated in outcome trials.

### Example 6: Platelet Endothelial Cell Adhesion Molecule-1 (PECAM-1) and Nitroxidative Stress Reduced by Eicosapentaenoic Acid (EPA) During Cytokine Exposure in Endothelial Cells

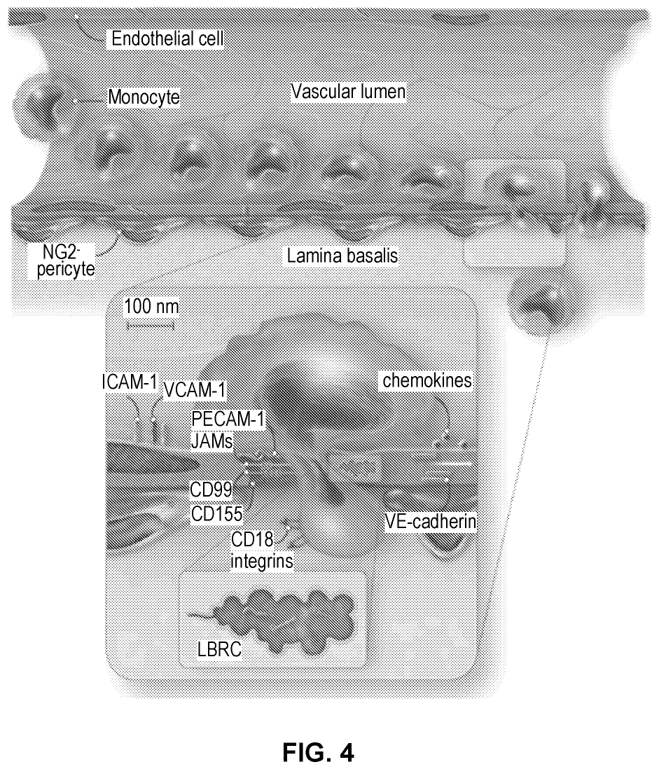

Platelet endothelial cell adhesion molecule 1 (PECAM-1) is a cell adhesion molecule constitutively expressed in endothelial cells (ECs), platelets, and leukocytes. Interaction between expressed PECAM-1 on leukocytes and ECs is required for transendothelial migration (TEM) under inflammatory conditions (FIG. 4). This protein, among others, is included in the Reactome database's “Neutrophil Degranulation” pathway, which can be analyzed using proteomic approaches. Previous studies have shown that challenging endothelial cells with interleukin-6 (IL-6) leads to endothelial dysfunction, which is characterized by overproduction of cytotoxic peroxynitrite (ONOO—).

The goal of the current study was to compare the effects of EPA and arachidonic acid (AA) on expression of pro-inflammatory proteins, including PECAM-1, and ONOO— in endothelial cells challenged with IL-6. The effects of EPA versus AA were compared on global protein expression, including PECAM-1, and correlated with peroxynitrite release from vascular ECs under inflammatory conditions with the cytokine IL-6.

Primary human umbilical vein endothelial cells (HUVECs) were purchased from Lonza (Manassas, VA). Cells were cultured in the recommended complete endothelial cell growth medium and maintained at 37° C. in a 95% air/5% CO2 humidified incubator. As recommended by the supplier, cells were supplied with fresh medium every other day and propagated by an enzymatic (trypsin) procedure as previously described. Cell culture medium also contained 2% FBS, which contained albumin to facilitate efficient delivery of fatty acid treatment. The fatty acids EPA and AA were purchased from Sigma-Aldrich (Saint Louis, MO) and solubilized in redistilled ethanol under nitrogen atmosphere. The various acid stock solutions were stored at −20° C. until use.

HUVECs were treated with vehicle, EPA, or AA (10 μM) for 2 hours, and then challenged with IL-6 (12 ng/ml) for 24 h hours. After incubation, cells were pelleted and frozen at −80° C. until proteomic analysis was performed. Cell pellets were lysed using methanol/chloroform extraction. Proteins were denatured, reduced, alkylated, and trypsin digested. Samples were then prepared for Tandem Mass Tag (TMT) 10plex labeling. A bicinchoninic acid (BCA) assay was performed on each sample to quantify the total protein in each sample, which is important to confirm equal amounts of each sample are added to the multiplex sample. Each peptide in a sample was given a unique, low molecular weight (typically 126-130 Da), and then the samples were combined. Relative protein expression levels among the various treatments were measured using LC/MS proteomic techniques. Following protein digest, peptides are separated over a reverse phase column and then identified based on their mass.

Each multiplexed sample was then fractionated to increase the overall protein coverage using high pH reversed phase fractionation and analyzed by LC/MS using a Dionex UltiMate 3000 RSLC in tandem with a Q-Exactive/Lumos Orbitrap Mass Spectrometer. The chromatography was performed using a 2-hour gradient on a Thermo Pepmap C18 column (100 Å pore size, 3.0 μm particle size, 100 μM×150 mm) set at 50° C. Mobile phase A was water with 0.1% formic acid, and mobile phase B was acetonitrile with 0.1% formic acid. Proteins that showed a fold change >1.0 and p<0.05 for the relevant comparisons were considered significant and further analyzed. Gene set enrichment analysis (GSEA) was also performed to assess changes in protein grouped by functional pathway based on the “Reactome” database.

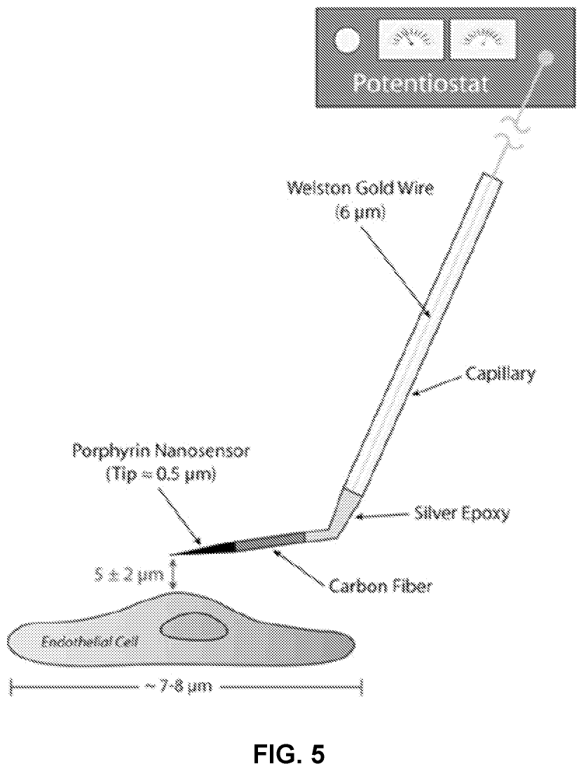

Release of ONOO— from HUVECs was measured as previously described. Briefly, in parallel with the cellular preparations for proteomic analysis, HUVECs were stimulated with calcium ionophore and ONOO— was measured using a porphyrinic nanosensor (FIG. 5).

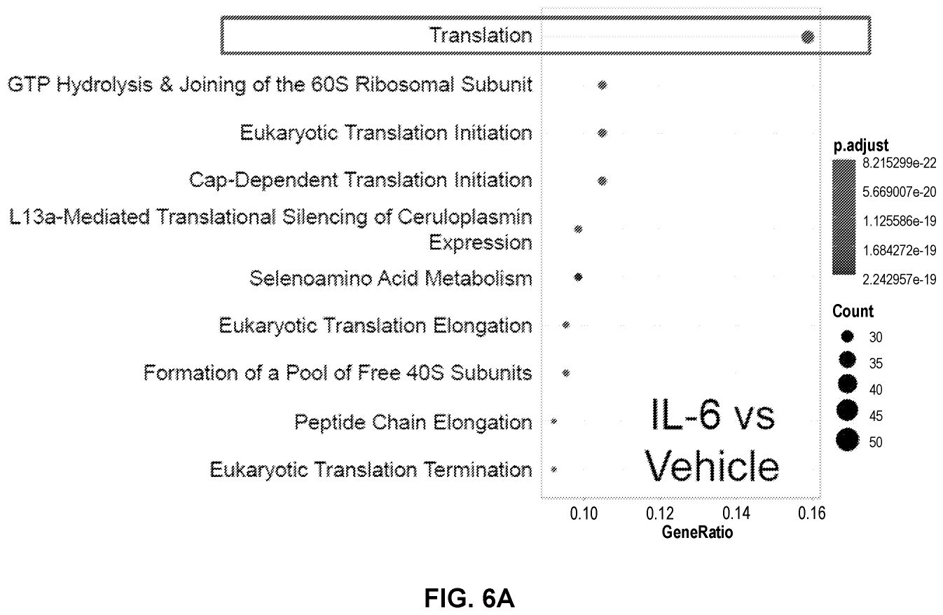

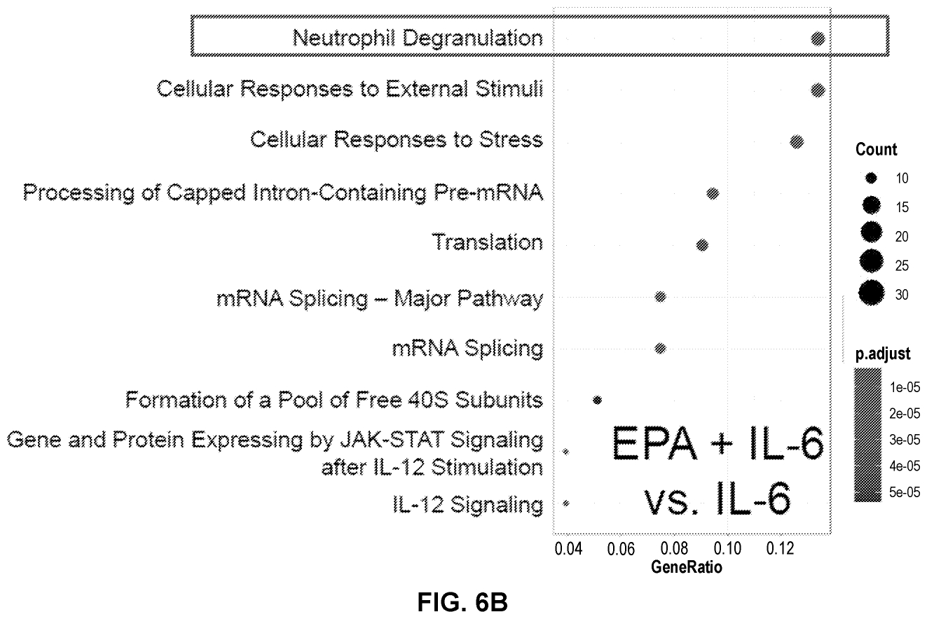

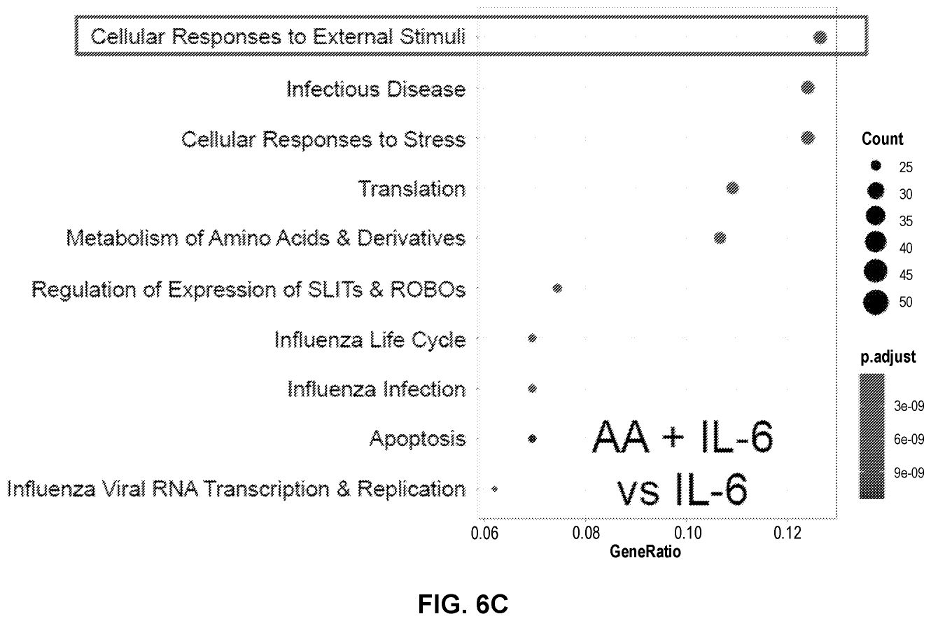

As shown in FIGS. 6A-6C, cells pretreated with EPA and AA significantly changed the expression of 327 and 549 proteins, respectively, compared with IL-6 alone. However, as shown in Table 3, only EPA caused a pronounced decrease in PECAM-1 by 120% (p=0.024). As shown in Table 4, EPA also significantly decreased ONOO— release by 17% (p=0.045) relative to IL-6 alone from the ECs unlike AA.

TABLE 3
Effects of EPA versus AA on PECAM-1
Expression Relative to IL-6

| Treatment | Fold Change Increase | P value |
|---|---|---|
| EPA + IL-6 vs. IL-6 | 1.22 | 0.024 |
| AA + IL-6 vs. IL-6 | — | — |

TABLE 4
Effects of EPA versus AA on Peroxynitrite
Release from HUVECs Changes with IL-6 (values
are mean ± SEM; N = 4-5)

| Treatment | ONOO- Release (nM) | Significance (vs. IL-6) |
|---|---|---|
| Vehicle | 174 ± 6 | p < 0.01 |
| IL-6 | 217 ± 10 | — |
| EPA + IL-6 | 181 ± 10 | p < 0.05 |
| AA + IL-6 | 194 ± 9 | n.s. |

Thus, these data show that EPA significantly reduced the expression of PECAM-1 as well as nitroxidative stress under conditions of inflammation. These anti-inflammatory effects of EPA in vascular endothelium were not reproduced by AA and may contribute to its CV benefits at pharmacologic levels in clinical trials.

### Example 7: A Rodent Model to Elucidate the Effects of Icosapent Ethyl on Tissue Heme Oxygenase Expression

The main objective of the present study is to test the hypothesis that various forms of eicosapentaenoic acid (EPA) can induce the expression of heme oxygenase (HO) and other selected genes of interest compared to control treatment among Long-Evans rats exposed to EPA by gavage daily for 21 days. It is biologically plausible that inducing HO and/or the other genes of interest could explain some of the therapeutic benefits of EPA.

### Eicosapentaenoic Acid

Icosapent ethyl (IPE) is an ethyl ester of EPA, an omega-3 long-chain polyunsaturated fatty acid, for which gram doses improves dyslipidemia and prevents major atherosclerotic vascular disease events.

Currently, a highly purified form of IPE, Vascepa, is used to treat hypertriglyceridemia and prevent major atherosclerotic vascular events (MACE).

### Heme Oxygenase

Separately, Heme Oxygenase 1 (HMOX1) is a Nrf2-regulated gene coding HO-1; the latter protein has important antioxidant, anti-inflammatory, antiapoptotic, and immunomodulatory effects in vascular cells and tissues, whereby HO-1 can mitigate adverse tissue injury responses (see FIG. 8).

Classically, HMOX1 is the inducible gene coding HO-1, induced by a variety of cell-injury and other stimuli. Other experiments suggest EPA can upregulate HMOX1 and HO-1. It is important to demonstrate this with IPE in particular, and with an intact organism model exposed to IPE by the GI route. If IPE induces HMOX1, this itself could mediate some of IPE's benefits, particularly for preventing MACE.

In contrast to HMOX1, Heme Oxygenase 2 (HMOX2) is separate gene with different regulators, encoding the protein HO-2. Importantly, HMOX2 is thought to be produced constitutively, and few molecules have been found that alter its expression. A notable exception to this is that HMOX2 is induced by corticosteroids. Steroid-induction might be mediated by suppressing arachidonic acid (ARA) oxidation, and in turn, limiting exposure to bioactive ARA-derived oxylipins, a canonical steroid effect. In the present experiment, Long-Evans rats were exposed to large daily doses of EPA. Such doses are also expected to diminish ARA oxidation, insofar as EPA competes with arachidonic acid and is metabolized to distinct, analogous oxylipins, many of which have anti-inflammatory effects. In other words, large-dose IPE may have anti-inflammatory effects via oxylipin metabolites that resemble critical aspects of corticosteroid effects. As such, IPE may offer an alternative to steroids to induce HMOX2.

The other genes of interest include genes that are co-regulated with HO-1 by the same antioxidant response element (ARE): NQ01 and GST. COX2 is an enzyme involved in converting EPA into bioactive oxylipins that may be less inflammatory compared to analogous oxylipins originating from ARA. Angiotensin-converting enzyme (ACE) is produced in the lungs and regulates angiotensin I to the vasoconstrictive angiotensin II, thereby affecting blood pressure. In unpublished proteomics work, ACE was inhibited by EPA.

### Scientific Approach

Preserved tissues from this study present an opportunity to test the extent to which IPE alone induces HO gene expression, compared to an oleic acid control (OA) and a combination of fatty acids similar to Oxepa®, a mixed omega-3 product used in sepsis and acute respiratory distress syndrome (ARDS).

Accordingly, this multiple dose response study will examine the effects of daily delivery for 21 days by gavage of various formulations of IPE on gene expression of HMOX1 and HMOX2, and related genes.

For reference, the 1.033 g IPE/kg/day dose in the Long-Evans rat is comparable to a 10 g/day dose in a human. Since Vascepa (i.e., ethyl EPA) is dosed at 4 g/day clinically, this is 2.5-fold the typical dose. This dose was selected as a reasonable dose to consider for the treatment of sepsis and ARDS. If IPE induces HMOX1, this too could have therapeutic benefits in the same conditions. As such, HMOX1 and/or HMOX2 induction by the same IPE doses would be biologically relevant.

Exemplary oxylipins useful for the study are listed below:
FA 20n5: Eicosapentaenoic Acid
Leukotriene B5
20-Hydroxy Leukotriene B5
Leukotriene C5
Leukotriene D5
Leukotriene E5
5-HEPE: 5-Hydroxy Eicosapentaenoic Acid
8-HEPE: 8-Hydroxy Eicosapentaenoic Acid
9-HEPE: 9-Hydroxy Eicosapentaenoic Acid
11-HEPE: 11-Hydroxy Eicosapentaenoic Acid
12-HEPE: 12-Hydroxy Eicosapentaenoic Acid
15-HEPE: 15-Hydroxy Eicosapentaenoic Acid
18-HEPE: 18-Hydroxy Eicosapentaenoic Acid
19-HEPE: 19-Hydroxy Eicosapentaenoic Acid
20-HEPE: 20-Hydroxy Eicosapentaenoic Acid
5-Oxo-EPE: 5-Keto Eicosapentaenoic Acid
12-Oxo-EPE: 12-Keto Eicosapentaenoic Acid
15-Oxo-EPE: 15-Keto Eicosapentaenoic Acid
LXA5: Lipoxin A5
Resolvin E1: 5,12,18-TriHydroxy Eicosapentaenoic Acid
Resolvin E4: 5,15-DiHydroxy Eicosapentaenoic Acid
5,6-DiHETE: 5,6-DiHydroxy Eicosatetraenoic Acid
8,9-DiHETE: 8,9-DiHydroxy Eicosatetraenoic Acid
11,12-DiHETE: 11,12-DiHydroxy Eicosatetraenoic Acid
14,15-DiHETE: 14,15-DiHydroxy Eicosatetraenoic Acid
17,18-DiHETE: 17,18-DiHydroxy Eicosatetraenoic Acid
5,6-EpETE: 5,6-Epoxy Eicosatetraenoic Acid
8,9-EpETE: 8,9-Epoxy Eicosatetraenoic Acid
11,12-EpETE: 11,12-Epoxy Eicosatetraenoic Acid
14,15-EpETE: 14,15-Epoxy Eicosatetraenoic Acid
17,18-EpETE: 17,18-Epoxy Eicosatetraenoic Acid
Prostaglandin D3
Prostaglandin E3
Prostaglandin F3α
2,3 dinor11β-Prostaglandin F3α
Prostaglandin A3
15d-PGJ3: 15-Deoxy-Δ12,14-Prostaglandin J3
Prostaglandin J3
Thromboxane B3
11-Dehydro-Thromboxane B3
6-Keto Prostaglandin F2α
RvE2: 5,18-DiHydroxy Eicosapentaenoic Acid
RvE3: 17,18-DiHydroxy Eicosapentaenoic Acid
8,18-DiHEPE: 8,18-DiHydroxy Eicosapentaenoic Acid
11,18-DiHEPE: 11,18-DiHydroxy Eicosapentaenoic Acid
12,18-DiHEPE: 12,18-DiHydroxy Eicosapentaenoic Acid

A total of 90 tissue samples will be analyzed for gene expression of HMOX1, HMOX2, and related genes. Expression in the tissues as outlined in the tables below. If results are positive, protein expression of HO-1 and/or HO-2 can be measured by Western blot in the same tissue samples. (see Collier J J, Batdorf H M, Merrifield K L, Martin T M, White U, Ravussin E, Burk D H, Cooley C R, Karlstad M D, Burke S J. Pioglitazone Reverses Markers of Islet Beta-Cell De-Differentiation in db/db Mice While Modulating Expression of Genes Controlling Inflammation and Browning in White Adipose Tissue from Insulin-Resistant Mice and Humans. Biomedicines. 2021; 9(9):1189).

Genes to be detected:

TABLE 5
Gene Expression Panel**

| Genes of Interest | |
|---|---|
| Detected | Associated Protein |
| HMOX1 | HO-1: heme oxygenase-1 |
| HMOX2 | HO-2: heme oxygenase-2 |
| NFE2L2 | Nrf2: nuclear factor erythroid 2-related factor 2 |
| COX2 | COX-2: cyclooxygenase 2 |
| NQ01 | NAD(P)H dehydrogenase [quinone] 1 |
| GST | Glutathione S-transferase |
| ACE (in lung tissue)* | ACE: angiotensin I converting enzyme |

Housekeeping/Control Genes:

| | |
|---|---|
| ACTB | beta-actin |
| 18S rRNA | 18S ribosomal RNA |

TABLE 6
Tissues Evaluated by Gene Expression Panel

| | Samples | | Total | | Tissues | | |
|---|---|---|---|---|---|---|---|
| Tissue | Collected | | Samples | | Assayed Per | Dietary | Total Tissue |
| Specimen | Per Rat | Rats | Collected | Assay | Dietary Group | Groups | Samples Assayed |
| Liver | 1 | 6 | 6 | Gene expression panel | 6 | 5 | 30 |
| Lung | 1 | 6 | 6 | Gene expression panel + | 6 | 5 | 30 |
| | | | | ACE (angiotensin I | | | |
| | | | | converting enzyme) | | | |
| Heart | 1 | 6 | 6 | Gene expression panel | 6 | 5 | 30 |

| | | | | |
|---|---|---|---|---|
| Totals | 18 | 18 | Totals | 90 |

### Gene Expression Analysis

Rat liver, lung and cardiac tissue will be powdered (liquid nitrogen) and 50 mg aliquots will be homogenized in TRIzol. Total ribonucleic acid (RNA) will be extracted from tissues using the RNeasy Mini RNA kit (Qiagen, Germantown, MD, USA). RNA quality and quantity will be assessed using a Nanodrop spectrophotometer (NanoDrop Technologies, Wilmington, DE, USA). Complementary deoxyribonucleic acid (cDNA) is generated from total RNA using the iScript cDNA synthesis kit (Bio-Rad). Relative messenger ribonucleic acid (mRNA) abundance is measured by real-time RT-PCR using the iTaq Universal SYBR Green Supermix (Bio-Rad) on a CFX96 instrument (Bio-Rad). Transcript levels are normalized to either housekeeping genes ACTB or 18sRNA. Primer pairs will be custom designed using the Primer3Plus software and DNA sequences purchased from IDT (1).

1. A method of treating or preventing endothelial dysfunction, increasing an activity of or an amount of heme oxygenase-1 (HO-1), and/or activating transcription factor Nrf2 (nuclear factor erythroid 2-related factor 2) in a subject, the method comprising administering to the subject a pharmaceutical composition comprising eicosapentaenoic acid (EPA) and/or a derivative thereof to provide a daily dose of about 1 g to about 20 g of the eicosapentaenoic acid and/or derivative thereof to the subject.

2. The method of claim 1, wherein the pharmaceutical composition comprises at least about 80%, at least about 90%, at least about 95%, or at least about 96%, by weight of all fatty acids present, the EPA and/or the derivative thereof.

3. The method of claim 1, wherein the pharmaceutical composition comprises no more than about 20%, no more than about 10%, no more than about 5%, or no more than about 3%, by weight of all fatty acids present, docosahexaenoic acid or derivatives thereof.

4. The method of claim 1, wherein the pharmaceutical composition comprises no docosahexaenoic acid or derivatives thereof.

5. The method of claim 1, wherein the subject is administered about 4 g, about 10 g, about 15 g, or about 20 g of the EPA and/or the derivative thereof per day.

6-8. (canceled)

9. The method of claim 1, wherein the subject is administered the pharmaceutical composition for a period of time between about 3 days to about 1 year.

10. The method of claim 1, wherein the EPA and/or the derivative thereof comprises eicosapentaenoic acid ethyl ester (E-EPA).

11. The method of claim 1, wherein the derivative of EPA is at least one selected from the group consisting of 6-keto-prostaglandin F2 alpha (6k-PGF2a), thromboxane B3 (TXB3), 11-dehydro-thromboxane B3 (11-dTXB3), prostaglandin F3 alpha (PGF3a), prostaglandin E3 (PGE3), prostaglandin A3 (PGA3), prostaglandin D3 (PGD3), 2,3-dinor 11 beta-prostaglandin F3 alpha (2,3-dinor11bPGF3a), prostaglandin J3 (PGJ3), 15-deoxy-delta-12,14-prostaglandin J3 (15d-PGJ3), leukotriene B5 (LTB5), 20-hydroxy-leukotriene B5 (20-OH-LTB5), leukotriene C5 (LTC5), leukotriene D5 (LTD5), leukotriene E5 (LTE5), lipoxin A5 (LXA5), 5-oxo-eicosapentaenoic acid (5-oxo-EPE), 12-oxo-eicosapentaenoic acid (12-oxo-EPE), 15-oxo-eicosapentaenoic acid (15-oxo-EPE), 5-hydroxyeicosapentaenoic acid (5-HEPE), 8-hydroxyeicosapentaenoic acid (8-HEPE), 9-hydroxyeicosapentaenoic acid (9-HEPE), 11-hydroxyeicosapentaenoic acid (11-HEPE), 12-hydroxyeicosapentaenoic acid (12-HEPE), 15-hydroxyeicosapentaenoic acid (15-HEPE), 18-hydroxyeicosapentaenoic acid (18-HEPE), 19-hydroxyeicosapentaenoic acid (19-HEPE), 20-hydroxyeicosapentaenoic acid (20-HEPE), 5,6-epoxyeicosatetraenoic acid (5,6-EpETE), 8,9-epoxyeicosatetraenoic acid (8,9-EpETE), 11,12-epoxyeicosatetraenoic acid (11,12-EpETE), 14,15-epoxyeicosatetraenoic acid (14,15-EpETE), 5,6-dihydroxyeicosatetraenoic acid (5,6-diHETE), 8,9-dihydroxyeicosatetraenoic acid (8,9-diHETE), 11,12-dihydroxyeicosatetraenoic acid (11,12-diHETE), 14,15-dihydroxyeicosatetraenoic acid (14,15-diHETE), 17,18-dihydroxyeicosatetraenoic acid (17,18-diHETE), 17,18-epoxyeicosatetraenoic acid (17,18-EpETE), Resolvin E1 (RvE1), Resolvin E2 (RvE2), Resolvin E3 (RvE3), and Resolvin E4 (RvE4).

12. The method of claim 1, wherein the pharmaceutical composition further comprises a polyunsaturated fatty acid or a derivative thereof which is chemically distinct from the EPA or the derivative thereof.

13. The method of claim 12, wherein the polyunsaturated fatty acid is a long-chain fatty acid (LCFA).

14. The method of claim 13, wherein the LCFA is a long-chain polyunsaturated fatty acid (LCPUFA).

15. The method of claim 12, wherein the polyunsaturated fatty acid or derivative thereof is at least one selected from the group consisting of linoleic acid (FA 18:2, or LA), arachidonic acid (FA 20:4, or AA), docosapentaenoic acid (FA 22:5, or DPA), docosahexaenoic acid (FA 22:6, or DHA), a linoleic acid derivative (LA derivative), an arachidonic acid derivative (AA derivative), and a docosahexaenoic acid derivative (DHA derivative).

16. The method of claim 15, wherein the LA derivative is 9-hydroxyoctadecadienoic acid (9-HODE), 13-hydroxyoctadecadienoic acid (13-HODE), or both and/or the AA derivative is at least one selected from the group consisting of 6-keto-prostaglandin F1 alpha (6k-PGF1a), thromboxane B2 (TXB2), 11-dehydro-thromboxane B2 (11-dTXB2), prostaglandin F2 alpha (PGF2a), prostaglandin E2 (PGE2), prostaglandin A2 (PGA2), prostaglandin D2 (PGD2), 2,3-dinor 11 beta-prostaglandin F2 alpha (2,3-dinor11bPGF2a), prostaglandin J2 (PGJ2), 15-deoxy-delta-12,14-prostaglandin J2 (15d-PGJ2), leukotriene B4 (LTB4), 20-hydroxy-leukotriene B4 (20-OH-LTB4), leukotriene C4 (LTC4), leukotriene D4 (LTD4), leukotriene E4 (LTE4), lipoxin A4 (LXA4), 5-oxo-eicosatetraenoic acid (5-oxo-ETE), 12-oxo-eicosatetraenoic acid (12-oxo-ETE), 15-oxo-eicosatetraenoic acid (15-oxo-ETE), 5-hydroxyeicosatetraenoic acid (5-HETE), 8-hydroxyeicosatetraenoic acid (8-HETE), 9-hydroxyeicosatetraenoic acid (9-HETE), 11-hydroxyeicosatetraenoic acid (11-HETE), 12-hydroxyeicosatetraenoic acid (12-HETE), 15-hydroxyeicosatetraenoic acid (15-HETE), 18-hydroxyeicosatetraenoic acid (18-HETE), 19-hydroxyeicosatetraenoic acid (19-HETE), 20-hydroxyeicosatetraenoic acid (20-HETE), 5,6-epoxyeicosatrienoic acid (5,6-EET), 8,9-epoxyeicosatrienoic acid (8,9-EET), 11,12-epoxyeicosatrienoic acid (11,12-EET), 14,15-epoxyeicosatrienoic acid (14,15-EET), 5,6-dihydroxyeicosatrienoic acid (5,6-diHET), 8,9-dihydroxyeicosatrienoic acid (8,9-diHET), 11,12-dihydroxyeicosatrienoic acid (11,12-diHET), 14,15-dihydroxyeicosatrienoic acid (14,15-diHET), and 12-hydroxyheptadecatrenoic acid (12-HHTrE).

17. (canceled)

18. The method of claim 15, wherein the DHA derivative is at least one selected from the group consisting of 14-hydroxydocosahexaenoic acid (14-HDoHE), 17-hydroxydocosahexaenoic acid (17-HDoHE), and Resolvin D1 (RvD1).

19. The method of claim 1, wherein administration of the pharmaceutical composition reduces platelet aggregation and/or inflammation in the subject.

20. (canceled)

21. The method of claim 1, wherein administration of the pharmaceutical composition increases nitric oxide (NO) bioavailability in the subject.

22. The method of claim 1, wherein administration of the pharmaceutical composition reduces a risk for thrombosis and/or atherosclerosis in the subject.

23-26. (canceled)

27. The method of claim 1, which activates antioxidant response elements (AREs) in the subject.

28. The method of claim 1, wherein administration of the pharmaceutical composition reduces a risk for or treats sepsis and/or acute respiratory distress syndrome (ARDS) in the subject.

29. (canceled)

30. A diagnostic method of assessing a suitability, dosage, and/or duration of the method of claim 1, the method comprising, prior to the administration, determining a concentration of HO-1 and/or a nucleotide sequence encoding HO-1, in bodily fluid or non-neural tissue obtained from the subject, and comparing the concentration with a corresponding concentration of HO-1 and/or an HO-1 encoding nucleotide sequence in a corresponding bodily fluid or non-neural tissue obtained from at least one control subject, wherein a reduced concentration of HO-1 and/or an HO-1 encoding nucleotide sequence in the subject compared to the control subject is used to determine the suitability, dosage, and/or duration.
